(19) United States
(12) Patent Application Publication
(10) Pub. No.: US 2026/0306568 A1
(43) Pub. Date: Oct. 8, 2026

(54) METHODS AND COMPOSITIONS OF BOTANICAL EXTRACTS

(71) Applicant: Unicity Properties, Inc., Provo, UT (US)

(72) Inventors: Dan Li, Singapore (SG); Daniel Hughes, Provo, UT (US)

(21) Appl. No.: 19/464,240

(22) Filed: Jan. 29, 2026

Related U.S. Application Data
(60) Provisional application No. 63/783,053, filed on Apr. 3, 2025.

(51) Int. Cl. A61K 36/185

(52) U.S. Cl. CPC A61K 36/185; A61K 2236/331; A61K 2236/37; A61K 2236/53

(57) ABSTRACT

Disclosed herein are methods of preparing a botanical extract, the botanical extract including saponins, polyphenols, and xanthines. Also provided herein are compositions including a botanical extract, the compositions formulated as a powder, tablet, capsule, or liquid, and methods of improving or treating a metabolic condition by administering a composition.

### INCORPORATION BY REFERENCE TO ANY PRIORITY APPLICATIONS

This application claims priority to U.S. Provisional Patent Application No. 63/783,053, filed Apr. 3, 2025, which is hereby incorporated by reference in its entirety.

### FIELD

The present disclosure relates generally to methods of preparing a botanical extract, the botanical extract including saponins, polyphenols, and xanthines. The present disclosure also relates to compositions including a botanical extract, the compositions formulated as a powder, tablet, capsule, or liquid. The present disclosure also relates to methods of improving or treating a metabolic condition by administering a composition.

### BACKGROUND

Bioactive compounds have long been extracted from plants, and the extracts used in a variety of pharmaceutical and nutraceutical products. Potent extracts are essential to effectively deliver bioactive compounds and impart diverse health benefits, as the concentration and quality of an extract's bioactive compounds directly influence its efficacy. These extracts and the extraction method must be carefully standardized to ensure high levels of key phytochemicals, such as polyphenols, xanthines, and saponins, which are responsible for the extract's antioxidant, metabolic, cognitive, and cardiovascular benefits.

Existing methods of extraction from plants include traditional hot water infusion, which offers simplicity but varies in effectiveness with the age of the plant's leaves and often results in a more bitter taste from older leaves due to higher tannin content (Janda et al., 2020; Najman et al., 2024). Cold water extraction, while preserving flavor, might not fully extract compounds from tougher older leaves, yielding less potent extracts (Blum-Silva et al., 2015). Solvent extractions like ethanol-water or supercritical CO₂ can target specific compounds but do not account for the biochemical differences between young and old leaves, which can lead to inconsistent potency or flavor profiles (Burris et al., 2012; Domingues et al., 2024; Hegel et al., 2021). Ultrasound-assisted extraction, though efficient, also does not leverage the unique profiles of different aged leaves for optimal extraction (Kozono et al., 2024; Schenk et al., 2021).

Optimized extraction methods that maintain the stability and bioavailability of bioactive compounds can be used to obtain potent extracts.

### SUMMARY

The present disclosure relates generally to methods of preparing a botanical extract, the botanical extract including saponins, polyphenols, and xanthines. The present disclosure also relates to compositions including a botanical extract, the compositions formulated as a powder, tablet, capsule, or liquid. The present disclosure also relates to methods of improving or treating a metabolic condition by administering a composition.

Some embodiments disclosed herein relate to method of preparing a botanical extract. In some embodiments, the methods include combining botanical leaves with a first solvent to form a first mixture, heating the first mixture, filtering the first mixture to obtain a first liquid extract and a first residual material, combining the first residual material with a second solvent to form a second mixture, heating the second mixture, filtering the second mixture to obtain a second liquid extract and a second residual material, and combining and concentrating the first liquid extract and second liquid extract to obtain the botanical extract.

In some embodiments, the botanical leaves are less than 6 months old. In some embodiments, the botanical leaves are 3-6 months old. In some embodiments, the botanical leaves are obtained from yerba mate. In some embodiments, the first solvent is water. In some embodiments, the first solvent to botanical leaves has a ratio ranging from about 1:1 to about 20:1 in mL to grams, such as 1:1, 2:1, 3:1, 4:1, 5:1, 6:1, 7:1, 8:1, 9:1, 10:1, 11:1, 12:1, 13:1, 14:1, 15:1, 16:1, 17:1 18:1, 19:1, or 20:1 or a ratio within an amount defined by any two of the aforementioned values. In some embodiments, the first mixture is heated to a temperature ranging from about 50° C. to about 70° C., such as 50, 55, 60, 65, or 70° C., or a temperature within an amount defined by any two of the aforementioned values. In some embodiments, the first mixture is heated for a time period of about 30 minutes.

In some embodiments, the second solvent is water. In some embodiments, the second solvent to first residual material has a ratio ranging from about 1:1 to about 10:1 in mL to grams, such as 1:1, 2:1, 3:1, 4:1, 5:1, 6:1, 7:1, 8:1, 9:1, or 10:1 or a ratio within an amount defined by any two of the aforementioned values. In some embodiments, the second mixture is heated to a temperature ranging from about 50° C. to about 70° C., such as 50, 55, 60, 65, or 70° C., or a temperature within an amount defined by any two of the aforementioned values. In some embodiments, the second mixture is heated for a time period of about 30 minutes.

In some embodiments, the methods include spray drying the botanical extract to form a botanical extract powder. In some embodiments, the botanical extract includes one or more saponins, one or more polyphenols, or one or more xanthines. In some embodiments, the botanical extract includes saponins in an amount of at least 2.5%, caffeoylquinic acids in an amount of at least 20%, and caffeine in an amount of at least 4%.

Some embodiments disclosed herein relate to compositions including a botanical extract. In some embodiments, the botanical extract includes at least 2.5% saponins, at least 20% polyphenols, and at least 4% xanthines. In some embodiments, the composition is formulated as a powder, tablet, capsule, or liquid.

In some embodiments, the botanical extract includes at least 2.75% saponins. In some embodiments, the botanical extract includes at least 3.5% saponins. In some embodiments, the botanical extract includes at least 21% polyphenols. In some embodiments, the botanical extract includes at least 25% polyphenols. In some embodiments, the polyphenols include one or more flavonoids, phenolic acids, stilbenes, and/or lignans. In some embodiments, the polyphenols include chlorogenic acids and/or flavonoids. In some embodiments, the chlorogenic acids include caffeoylquinic acids, dicaffeoylquinic acids, feruloylquinic acids, cumaroylquinic acids, or a derivative thereof, or any combinations thereof. In some embodiments, the chlorogenic acids include caffeoylquinic acid.

In some embodiments, the botanical extract includes at least 5% xanthines. In some embodiments, the xanthines include one or more aminophylline, 3-isobutyl-1-methylxanthine, paraxanthine, pentoxyifylline, diprophylline, uric acid, theophylline, caffeine, theobromine, and/or 7-methylxanthine, or derivatives or combinations thereof. In some embodiments, the xanthines include caffeine.

In some embodiments, the botanical extract has a (1,1-diphenyl-2-picrylhydrazyl) (DPPH) IC₅₀ value of less than 1,000 μg/mL, such as less than 1000, 900, 800, 700, 600, 500, 400, 300, 200, or 100 μg/mL, or an amount less than any of the aforementioned values. In some embodiments, the botanical extract has a (1,1-diphenyl-2-picrylhydrazyl) (DPPH) IC₅₀ value of less than 207 μg/mL. In some embodiments, the botanical extract has an α-glucosidase IC₅₀ value of less than 200 g/mL, such as less than 200, 190, 180, 170, 160, 150, 140, 130, 120, 110, 100, or 50 μg/mL, or an amount less than any of the aforementioned values. In some embodiments, the botanical extract has an α-glucosidase IC₅₀ value of less than 160 μg/mL.

In some embodiments, the botanical extract includes one or more amino acids. In some embodiments, the one or more amino acids comprises histidine, isoleucine, leucine, lysine, methionine, phenylalanine, threonine, tryptophane, or valine. In some embodiments, the botanical extract includes at least 45 mg threonine per 100 g of extract. In some embodiments, the botanical extract comprises at least 45 mg tryptophan per 100 g of extract. In some embodiments, the botanical extract includes at least 1300 mg magnesium per 100 g of extract. In some embodiments, the botanical extract includes at least 4 mg niacin per 100 g of extract.

In some embodiments, the botanical extract has an oxygen radical absorbance capacity (ORAC) value of at least 170,000 μmol trolox equivalents (TE) per 100 g of extract. In some embodiments, the botanical extract has an ORAC value of at least 450,000 μmol TE per 100 g of extract.

In some embodiments, the botanical extract is prepared from botanical leaves that are 2 years or younger. In some embodiments, the botanical extract is an extract of botanical leaves that are 1 year or younger. In some embodiments, the botanical extract is prepared from botanical leaves that are 6 months or younger. In some embodiments, the botanical extract is prepared from botanical leaves that are about 3-6 months old.

In some embodiments, the botanical leaves are yerba mate leaves. In some embodiments, the botanical extract has elevated antioxidant activity. In some embodiments, the composition is formulated as a powder. In some embodiments, the composition is formulated as an antioxidant supplement. In some embodiments, the composition is formulated as a food product.

Some embodiments disclosed herein relate to methods of improving or treating a metabolic condition by administering to a subject in need a composition. In some embodiments, the composition includes a botanical extract. In some embodiments, the botanical extract includes at least 2.5% saponins, at least 20% polyphenols, and at least 4% xanthines. In some embodiments, the composition in formulated as a powder, tablet, capsule, or liquid.

In some embodiments, the metabolic condition is excess body weight, diabetes, insulin resistance, hyperglycemia, or any combination thereof. In some embodiments, improving or treating a metabolic condition includes reducing body weight, improving glycemic control, reducing insulin resistance, or any combination thereof. In some embodiments, the composition is configured to enhance alpha-glucosidase inhibitory effect, increase bioactivity, reduce insulin resistance, improve glycemic control, or modulate carbohydrate metabolism.

In some embodiments, the composition includes at least 1 mg of saponins. In some embodiments, the composition includes at least 600 mg of total chlorogenic acids. In some embodiments, the composition includes at least 60-180 mg of caffeine.

In some embodiments, the composition is formulated as a powder, antioxidant supplement, or a food product. In some embodiments, the composition is formulated for oral ingestion. In some embodiments, the composition is administered orally at least once daily.

### DETAILED DESCRIPTION

The present disclosure can be understood more readily by referencing the following detailed description, examples, and claims, and their previous and following descriptions. However, before the present compositions and methods are disclosed and described, it is to be understood that this disclosure is not limited to the specific compositions and methods disclosed unless otherwise specified. It is also to be understood that the terminology used herein is for the purpose of describing particular aspects only and is not necessarily intended to be limiting.

The description is provided as an enabling teaching of the disclosure. To this end, those skilled in the relevant art will recognize and appreciate that many changes can be made to the various aspects of the disclosure described herein while still obtaining beneficial results. It will also be apparent that some of the desired benefits can be obtained by selecting some of the features described herein without utilizing other features. Accordingly, those who work in the art will recognize that many modifications and adaptations to the present description are possible and can even be desirable in certain circumstances and are a part of the present disclosure. Thus, this description is provided as illustrative of certain principles of the present disclosure and not in limitation thereof.

As summarized herein, aspects of the methods of preparing a botanical extract, compositions and methods of improving or treating a metabolic condition using compositions including a botanical extract are provided herein. The compositions and methods provided herein exhibit several advantages over existing compositions and methods, including superior potency and effectiveness. In particular, embodiments of compositions and methods described herein use young yerba mate leaves for extraction of high concentrations of beneficial compounds, including, for example, antioxidants and chlorogenic acids, which result in extracts with superior potency. In particular, young leaves may be used to extract high concentrations of phytochemicals, including polyphenols, which are formulated into compositions to provide health benefits, such as improved lipid profiles and support for weight management. Advantageously, extracts from young leaves have a milder, more pleasant flavor, which can enhance appeal and acceptance. In addition, the compositions and methods of preparing a botanical extract disclosed herein ensures consistent quality and heightened potency.

In some embodiments, the use of young leaves eliminates the need for harsh chemicals or extreme conditions that could degrade the extract's quality. Harvesting young leaves also promotes sustainable agricultural practices, as they regenerate more quickly, and creates economic opportunities by enabling the production of premium yerba mate products. These advantages not only enhance the potency of the extract but also simplify the extraction process.

Embodiments of the methods of preparing a botanical extract disclosed herein relate to age-specific leaf biochemistry due to synergistic interaction of saponins, CQAs, caffeine, and nutritional components in young leaves, enhancing α-glucosidase inhibition and antioxidant capacity beyond individual compound effects, as evidenced by peak bioactivity and ORAC potency. Embodiments of the methods disclosed herein optimize the extract's therapeutic potential for managing diabetes (via α-glucosidase inhibition), supporting metabolic health, providing antioxidant protection (via ORAC-mediated peroxyl radical neutralization), and promoting muscle, bone, immune, and cognitive health. Some embodiments of the methods disclosed herein use peak defensive and metabolic activity of young leaves, resulting in a high-potency botanical extract distinct from extracts derived from older leaves with diminished bioactivity and nutrition.

It is to be understood that this disclosure is not limited to particular embodiments described, as such may, of course, vary. It is also to be understood that the terminology used herein is for the purpose of describing particular embodiments only, and is not intended to be limiting.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

As used herein, the term “botanical extract” has its ordinary meaning as commonly understood by those skilled in the art, and includes a process of removing material, compounds, compositions, or other components from a starting plant material. A process of extraction can be performed by various means, including by physical or chemical extraction, such as by pressing, grinding, heating, stirring, or other known methods for extracting a component from a starting material.

As used herein, the terms “young leaves” is understood to mean leaves that are in an early stage of development with respect to the botanical, including leaves that have recently emerged or are in early stages of growth. In some embodiments, young leaves include leaves that are typically smaller, softer, and lighter in color as compared to mature leaves. For fast growing plants, young leaves may include leaves from a few days to a couple of weeks old, and in slow growing plants, young leaves may include leaves from a few weeks to many months in age. For example, for yerba mate, young leaves can include leaves that are less than about 2 years old, for example, less than about 24, 23, 22, 21, 20, 19, 18, 17, 16, 15, 14, 13, 12, 11, 10, 9, 8, 7, 6, 5, 4, 3, 2, or 1 month old. In some embodiments, young leaves from yerba mate are about 3-6 months old, also known as “yerba mate green leaves.”

As used herein, the term “saponins” has its ordinary meaning as commonly understood by those skilled in the art and is understood to mean naturally occurring compounds found primarily in plants that are a subclass of terpenoids and are either triterpene glycosides or steroid glycosides.

As used herein, the term “polyphenols” has its ordinary meaning as commonly understood by those skilled in the art and is understood to mean naturally occurring compounds found in plants that are characterized by the presence of more than one phenol units. As used herein, the term “phenol” has its ordinary meaning as commonly understood by those skilled in the art and is understood to mean a benzene ring containing hydroxyl group(s). Polyphenols include flavonoids, phenolic acids, stilbenes, and lignans.

As used herein, the term “flavonoids” has its ordinary meaning as commonly understood by those skilled in the art and is understood to mean compounds that are characterized by a diphenylpropane structure of two benzene rings connected by a three-carbon chain that forms a pyran ring, as follows:

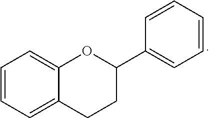 [SMILES: c1ccc(C2CCc3ccccc3O2)cc1]

Flavonoids include flavonols, flavones, isoflavones, anthocyanidins, flavanones, flavanols, and chalcones.

As used herein, the term “phenolic acids” has its ordinary meaning as commonly understood by those skilled in the art and is understood to mean compounds that are characterized by a phenol structure with carboxylic acid group(s). Phenolic acids include hydroxybenzoic acids and hydroxycinnamic acids. Hydroxybenzoic acids include gallic acids, protocatechuic acids, p-hydroxybenzoic acids, and syringic acids. Hydroxycinnamic acids include chlorogenic acids, p-coumaric acids, caffeic acids, ferulic acids, and sinapic acids.

As used herein, the term “stilbenes” has its ordinary meaning as commonly understood by those skilled in the art and is understood to mean compounds that are characterized by a 1,2-diphenylethylene structure. Stilbenes include glycoside stilbenes and aglycone stilbenes.

As used herein, the term “lignans” has its ordinary meaning as commonly understood by those skilled in the art. Lignans include furofuran lignans, furan lignans, dibenzylbutane lignans, dibenzylbutyrolacton lignans, aryltetralin lignans, arylnaphthalene lignans, dibenzocyclooctadiene lignans, and dibenzylbutyrolactol lignans.

As used herein, the term “xanthines” has its ordinary meaning as commonly understood by those skilled in the art and is understood to mean compounds that are characterized by a double ring structure including a pyrimidine ring and an imidazole ring. Xanthines include methylxanthines.

As used herein, the term “methylxanthines” has its ordinary meaning as commonly understood by those skilled in the art and is understood to mean compounds that are characterized by a double ring structure including a pyrimidine ring and an imidazole ring, possessing at least one methyl functional group. Methylxanthines include aminophylline, 3-isobutyl-1-methylxanthine, paraxanthine, pentoxyifylline, diprophylline, uric acid, theophylline, caffeine, theobromine, and 7-methylxanthine, and derivatives thereof.

As used herein, the term “caffeoylquinic acids” is understood to mean caffeoylquinic acids and di-caffeoylquinic acids, for example 3-CQA, 4-CQA, 5-CQA, 3,4-diCQA, 3,5-diCQA, and 4,5-diCQA).

### Methods of Preparing a Botanical Extract

Some embodiments disclosed herein relate to methods of preparing a botanical extract. In some embodiments, the methods include combining botanical leaves with a first solvent to form a first mixture, heating the first mixture, filtering the first mixture to obtain a first liquid extract and a first residual material, combining the first residual material with a second solvent to form a second mixture, heating the second mixture, filtering the second mixture to obtain a second liquid extract and a second residual material, and combining and concentrating the first liquid extract and second liquid extract to obtain the botanical extract.

In some embodiments, the botanical leaves are less than 6 months old, such as 1, 2, 3, 4, 5, or 6 months old, or an age within a range defined by any two of the aforementioned values. In some embodiments, the botanical leaves are 3-6 months old. In some embodiments, the botanical leaves are obtained from yerba mate. In some embodiments, the first solvent is water. In some embodiments, the first solvent to botanical leaves has a ratio ranging from about 1:1 to about 20:1 in mL to grams, such as 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1, or a ratio within a range defined by any two of the aforementioned values. In some embodiments, the first solvent to botanical leaves has a ratio of 10:1 in mL to grams. In some embodiments, the first mixture is heated to a temperature ranging from about 50° C. to about 70° C., such as 50, 55, 60, 65, or 70° C., or a temperature within an amount defined by any two of the aforementioned values. In some embodiments, the first mixture is heated to a temperature from about 55° C. to about 60° C. In some embodiments, the first mixture is heated for a time period of about 30 minutes, such as 20, 25, 30, 35, or 40 minutes, or any amount of time within a range defined by any two of the aforementioned values.

In some embodiments, the second solvent is water. In some embodiments, the second solvent to first residual material has a ratio ranging from about 1:1 to about 10:1 in mL to grams, such as 1:1, 2:1, 3:1, 4:1, 5:1, 6:1, 7:1, 8:1, 9:1, or 10:1, or a ratio within a range defined by any two of the aforementioned values. In some embodiments, the first solvent to botanical leaves has a ratio of 5:1 in mL to grams. In some embodiments, the second mixture is heated to a temperature ranging from about 50° C. to about 70° C., such as 50, 55, 60, 65, or 70° C., or a temperature within an amount defined by any two of the aforementioned values. In some embodiments, the first mixture is heated to a temperature from about 55° C. to about 60° C. In some embodiments, the second mixture is heated for a time period of about 30 minutes, such as 20, 25, 30, 35, or 40 minutes, or any amount of time within a range defined by any two of the aforementioned values.

In some embodiments, the methods include a first extraction and a second extraction. In some embodiments, the first extraction includes combining botanical leaves with a first solvent to form a first mixture, heating the first mixture, filtering the first mixture to obtain a first liquid extract and a first residual material. In some embodiments, the second extraction includes combining the first residual material with a second solvent to form a second mixture, heating the second mixture, filtering the second mixture to obtain a second liquid extract and a second residual material. In some embodiments the second extraction targets compounds resistant to the first extraction. In some embodiments, the methods include spray drying the botanical extract to form a botanical extract powder. In some embodiments, the botanical extract includes one or more saponins, one or more polyphenols, or one or more xanthines, or any combination thereof.

In some embodiments, the botanical extract includes at least 2.5% saponins, such as 2.5, 2.6, 2.7, 2.8, 2.9, 3.0, 3.1, 3.2, 3.3, 3.4, 3.5, 3.6, 3.7, 3.8, 3.9, or 4.0%, or any percent within a range defined by any two of the aforementioned values. In some embodiments, the botanical extract includes at least 2.75% saponins. In some embodiments, the botanical extract includes at least 3.5% saponins.

In some embodiments, the botanical extract includes at least 20% polyphenols, such as 20.0, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5, or 30.0%, or any percent within a range defined by any two of the aforementioned values. In some embodiments, the botanical extract includes at least 21% polyphenols. In some embodiments, the botanical extract includes at least 25% polyphenols. In some embodiments, the polyphenols include flavonoids, phenolic acids, stilbenes, and/or lignans. In some embodiments, the flavonoids include flavonols, flavones, isoflavones, anthocyanidins, flavanones, flavanols, and chalcones. In some embodiments, the phenolic acids include hydroxybenzoic acids and hydroxycinnamic acids. In some embodiments, the hydroxybenzoic acids include gallic acids, protocatechuic acids, p-hydroxybenzoic acids, and syringic acids. In some embodiments, the hydroxycinnamic acids include chlorogenic acids, p-coumaric acids, caffeic acids, ferulic acids, and sinapic acids. In some embodiments, the stilbenes include glycoside stilbenes and aglycone stilbenes. In some embodiments, the lignans include furofuran lignans, furan lignans, dibenzylbutane lignans, dibenzylbutyrolacton lignans, aryltetralin lignans, arylnaphthalene lignans, dibenzocyclooctadiene lignans, and dibenzylbutyrolactol lignans.

In some embodiments, the polyphenols include chlorogenic acids or flavonoids. In some embodiments, the chlorogenic acids include caffeoylquinic acids, dicaffeoylquinic acids, feruloylquinic acids, cumaroylquinic acids, or a derivative thereof, or any combinations thereof. In some embodiments, the chlorogenic acids include caffeoylquinic acid (including, for example, 3-CQA, 4-CQA, 5-CQA, 3,4-diCQA, 3,5-diCQA, and 4,5-diCQA). In some embodiments, the flavonoids include flavonols. In some embodiments the flavonols include rutin.

In some embodiments, the botanical extract includes at least 4% xanthines, such as 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0% or any percent within a range defined by any two of the aforementioned values. In some embodiments, the xanthines include methylxanthines. In some embodiments, the botanical extract includes at least 5% xanthines. In some embodiments, the xanthines are aminophylline, 3-isobutyl-1-methylxanthine, paraxanthine, pentoxyifylline, diprophylline, uric acid, theophylline, caffeine, theobromine, or 7-methylxanthine, or derivatives or combinations thereof. In some embodiments, the xanthines are caffeine.

In some embodiments, the botanical extract has a (1,1-diphenyl-2-picrylhydrazyl) (DPPH) IC₅₀ value of less than 1 mg/mL. In some embodiments, the botanical extract has a (1,1-diphenyl-2-picrylhydrazyl) (DPPH) IC₅₀ value of less than 207 μg/mL, such as 206, 205, 204, 203, 202, 201, 200, 199, 195, or 190 μg/mL, or a value within a range defined by any two of the aforementioned values. In some embodiments, the botanical extract has an α-glucosidase IC₅₀ value of less than 200 μg/mL, such as 200, 190, 180, 170, 160, or 150 μg/mL, or any value within a range defined by any two of the aforementioned values. In some embodiments, the botanical extract has an α-glucosidase IC₅₀ value of less than 160 μg/mL.

In some embodiments, the botanical extract includes one or more amino acids. In some embodiments, the one or more amino acids comprises histidine, isoleucine, leucine, lysine, methionine, phenylalanine, threonine, tryptophane, and/or valine. In some embodiments the one or more amino acids further include alanine, arginine, cystine, glutamic acid, glycine, proline, serine, and/or tyrosine. In some embodiments, the botanical extract includes at least 10 mg amino acids per 100 g of extract, such as 10, 50, 100, 200, 300, 400, 500, 600, 700, 800, 900, or 1000 mg or any value within a range defined by any two of the aforementioned values.

In some embodiments, the botanical extract includes at least 15 mg histidine per 100 g of extract, such as 15, 20, or 25 mg, or any value within a range defined by any two of the aforementioned values. In some embodiments, the botanical extract includes at least 25 mg isoleucine per 100 g of extract, such as 25, 30, or 35 mg, or any value within a range defined by any two of the aforementioned values. In some embodiments, the botanical extract includes at least 35 mg leucine per 100 g of extract, such as 35, 40, 45, or 50 mg, or any value within a range defined by any two of the aforementioned values. In some embodiments, the botanical extract includes at least 40 mg lysine per 100 g of extract, such as 40, 45, 50, 55, or 60 mg, or any value within a range defined by any two of the aforementioned values. In some embodiments, the botanical extract includes at least 10 mg methionine per 100 g of extract, such as 10, 12.5, or 15 mg, or any value within a range defined by any two of the aforementioned values. In some embodiments, the botanical extract includes at least 25 mg phenylalanine per 100 g of extract, such as 25, 30, or 35 mg, or any value within a range defined by any two of the aforementioned values. In some embodiments, the botanical extract includes at least 20 mg threonine per 100 g of extract, such as 20, 30, 40, or 50 mg, or any value within a range defined by any two of the aforementioned values. In some embodiments, the botanical extract includes at least 45 mg threonine per 100 g of extract. In some embodiments, the botanical extract includes at least 20 mg tryptophan per 100 g of extract, such as 20, 30, 40, or 50 mg, or any value within a range defined by any two of the aforementioned values. In some embodiments, the botanical extract includes at least 45 mg tryptophan per 100 g of extract. In some embodiments, the botanical extract includes at least 30 mg valine per 100 g of extract, such as 30, 40, or 50 mg, or any value within a range defined by any two of the aforementioned values.

In some embodiments, the botanical extract includes minerals. In some embodiments the minerals include calcium, copper, iron, magnesium, manganese, phosphorus, potassium, sodium, and/or zinc. In some embodiments, the botanical extract includes at least 0.5 mg minerals per 100 g of extract, such as 0.5, 1, 10, 50, 100, 500, 1000, 2000, or 5000 mg or any value within a range defined by any two of the aforementioned values. In some embodiments, the botanical extract includes at least 1300 mg magnesium per 100 g of extract.

In some embodiments, the botanical extract includes B-Vitamins. In some embodiments, the B-Vitamins include Vitamin B1 (thiamine), Vitamin B2 (riboflavin), Vitamin B3 (niacin), Vitamin B5 (pantothenic acid), Vitamin B6 (pyridoxine), and Vitamin B7 (biotin). In some embodiments, the botanical extract includes at least 0.1 mg B-Vitamins per 100 g of extract, such as 0.1, 0.5, 1, 2, 3, 4, 5, or 6 mg, or any value within a range defined by any two of the aforementioned values. In some embodiments, the botanical extract includes at least 4 mg niacin per 100 g of extract, such as 1, 2, 3, 4, or 5 mg, or any value within a range defined by any two of the aforementioned values.

In some embodiments, the botanical extract has an oxygen radical absorbance capacity (ORAC) value of at least 170,000 μmol trolox equivalents (TE) per 100 g of extract, such as 190,000, 200,000, 210,000, 220,000, 230,000, 240,000, 250,000, 260,000, 270,000, 280,000, 290,000, 300,000, 310,000, 320,000, 330,000, 340,000, 350,000, 360,000, 370,000, 380,000, 390,000, 400,000, 410,000, 420,000, 430,000, 440,000, 450,000, or 460,000 μmol trolox equivalents (TE) per 100 g of extract, or any value within a range defined by any two of the aforementioned values. In some embodiments, the botanical extract has an ORAC value of at least 450,000 μmol TE per 100 g of extract.

In some embodiments, the botanical extract is prepared from botanical leaves that are 2 years or younger. In some embodiments, the botanical extract is an extract of botanical leaves that are 1 year or younger. In some embodiments, the botanical extract is prepared from botanical leaves that are 6 months or younger. In some embodiments, the botanical extract is prepared from botanical leaves that are about 3-6 months old.

In some embodiments, the botanical leaves are yerba mate leaves. In some embodiments, the botanical extract has elevated antioxidant activity. In some embodiments, the composition is formulated as a powder. In some embodiments, the composition is formulated as an antioxidant supplement. In some embodiments, the composition is formulated as a food product.

In some embodiments, the methods disclosed herein can be used to prepare any of the botanical extracts disclosed herein. In some embodiments, the methods disclosed herein can be used to prepare any of the botanical extracts included in any of the compositions disclosed herein. In some embodiments, the methods disclosed herein can be used to prepare any of the botanical extracts used in any of the methods of treating or improving a metabolic condition disclosed herein.

### Compositions Including a Botanical Extract

Some embodiments disclosed herein relate to compositions including a botanical extract. In some embodiments, the botanical extract includes one or more saponins, one or more polyphenols, and one or more xanthines. In some embodiments, the composition is formulated as a powder, tablet, capsule, or liquid.

In some embodiments, the botanical extract includes at least 2.5% saponins, such as 2.5, 2.6, 2.7, 2.8, 2.9, 3.0, 3.1, 3.2, 3.3, 3.4, 3.5, 3.6, 3.7, 3.8, 3.9, or 4.0%, or any percent within a range defined by any two of the aforementioned values. In some embodiments, the botanical extract includes at least 2.75% saponins. In some embodiments, the botanical extract includes at least 3.5% saponins.

In some embodiments, the botanical extract includes at least 20% polyphenols, such as 20.0, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5, or 30.0%, or any percent within a range defined by any two of the aforementioned values. In some embodiments, the botanical extract includes at least 21% polyphenols. In some embodiments, the botanical extract includes at least 25% polyphenols. In some embodiments, the polyphenols include flavonoids, phenolic acids, stilbenes, and/or lignans. In some embodiments, the flavonoids include flavonols, flavones, isoflavones, anthocyanidins, flavanones, flavanols, and chalcones. In some embodiments, the phenolic acids include hydroxybenzoic acids and hydroxycinnamic acids. In some embodiments, the hydroxybenzoic acids include gallic acids, protocatechuic acids, p-hydroxybenzoic acids, and syringic acids. In some embodiments, the hydroxycinnamic acids include chlorogenic acids, p-coumaric acids, caffeic acids, ferulic acids, and sinapic acids. In some embodiments, the stilbenes include glycoside stilbenes and aglycone stilbenes. In some embodiments, the lignans include furofuran lignans, furan lignans, dibenzylbutane lignans, dibenzylbutyrolacton lignans, aryltetralin lignans, arylnaphthalene lignans, dibenzocyclooctadiene lignans, and dibenzylbutyrolactol lignans.

In some embodiments, the polyphenols include chlorogenic acids or flavonoids. In some embodiments, the chlorogenic acids include caffeoylquinic acids, dicaffeoylquinic acids, feruloylquinic acids, cumaroylquinic acids, or a derivative thereof, or any combinations thereof. In some embodiments, the chlorogenic acids include caffeoylquinic acid (including, for example, 3-CQA, 4-CQA, 5-CQA, 3,4-diCQA, 3,5-diCQA, and 4,5-diCQA). In some embodiments, the flavonoids include flavonols. In some embodiments the flavonols include rutin.

In some embodiments, the botanical extract includes at least 4% xanthines, such as 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0% or any percent within a range defined by any two of the aforementioned values. In some embodiments, the xanthines include methylxanthines. In some embodiments, the botanical extract includes at least 5% xanthines. In some embodiments, the xanthines are aminophylline, 3-isobutyl-1-methylxanthine, paraxanthine, pentoxyifylline, diprophylline, uric acid, theophylline, caffeine, theobromine, or 7-methylxanthine, or derivatives or combinations thereof. In some embodiments, the xanthines are caffeine.

In some embodiments, the botanical extract has a (1,1-diphenyl-2-picrylhydrazyl) (DPPH) IC₅₀ value of less than 1 mg/mL. In some embodiments, the botanical extract has a (1,1-diphenyl-2-picrylhydrazyl) (DPPH) IC₅₀ value of less than 207 μg/mL, such as 206, 205, 204, 203, 202, 201, 200, 199, 195, or 190 μg/mL, or a value within a range defined by any two of the aforementioned values. In some embodiments, the botanical extract has an α-glucosidase IC₅₀ value of less than 200 μg/mL, such as 200, 190, 180, 170, 160, or 150 μg/mL, or any value within a range defined by any two of the aforementioned values. In some embodiments, the botanical extract has an α-glucosidase IC₅₀ value of less than 160 μg/mL.

In some embodiments, the botanical extract includes one or more amino acids. In some embodiments, the one or more amino acids comprises histidine, isoleucine, leucine, lysine, methionine, phenylalanine, threonine, tryptophane, and/or valine. In some embodiments the one or more amino acids further include alanine, arginine, cystine, glutamic acid, glycine, proline, serine, and/or tyrosine. In some embodiments, the botanical extract includes at least 10 mg amino acids per 100 g of extract, such as 10, 50, 100, 200, 300, 400, 500, 600, 700, 800, 900, or 1000 mg or any value within a range defined by any two of the aforementioned values.

In some embodiments, the botanical extract includes at least 15 mg histidine per 100 g of extract, such as 15, 20, or 25 mg, or any value within a range defined by any two of the aforementioned values. In some embodiments, the botanical extract includes at least 25 mg isoleucine per 100 g of extract, such as 25, 30, or 35 mg, or any value within a range defined by any two of the aforementioned values. In some embodiments, the botanical extract includes at least 35 mg leucine per 100 g of extract, such as 35, 40, 45, or 50 mg, or any value within a range defined by any two of the aforementioned values. In some embodiments, the botanical extract includes at least 40 mg lysine per 100 g of extract, such as 40, 45, 50, 55, or 60 mg, or any value within a range defined by any two of the aforementioned values. In some embodiments, the botanical extract includes at least 10 mg methionine per 100 g of extract, such as 10, 12.5, or 15 mg, or any value within a range defined by any two of the aforementioned values. In some embodiments, the botanical extract includes at least 25 mg phenylalanine per 100 g of extract, such as 25, 30, or 35 mg, or any value within a range defined by any two of the aforementioned values. In some embodiments, the botanical extract includes at least 20 mg threonine per 100 g of extract, such as 20, 30, 40, or 50 mg, or any value within a range defined by any two of the aforementioned values. In some embodiments, the botanical extract includes at least 45 mg threonine per 100 g of extract. In some embodiments, the botanical extract includes at least 20 mg tryptophan per 100 g of extract, such as 20, 30, 40, or 50 mg, or any value within a range defined by any two of the aforementioned values. In some embodiments, the botanical extract includes at least 45 mg tryptophan per 100 g of extract. In some embodiments, the botanical extract includes at least 30 mg valine per 100 g of extract, such as 30, 40, or 50 mg, or any value within a range defined by any two of the aforementioned values.

In some embodiments, the botanical extract includes minerals. In some embodiments the minerals include calcium, copper, iron, magnesium, manganese, phosphorus, potassium, sodium, and/or zinc. In some embodiments, the botanical extract includes at least 0.5 mg minerals per 100 g of extract, such as 0.5, 1, 10, 50, 100, 500, 1000, 2000, or 5000 mg or any value within a range defined by any two of the aforementioned values. In some embodiments, the botanical extract includes at least 1300 mg magnesium per 100 g of extract.

In some embodiments, the botanical extract includes B-Vitamins. In some embodiments, the B-Vitamins include Vitamin B1 (thiamine), Vitamin B2 (riboflavin), Vitamin B3 (niacin), Vitamin B5 (pantothenic acid), Vitamin B6 (pyridoxine), and Vitamin B7 (biotin). In some embodiments, the botanical extract includes at least 0.1 mg B-Vitamins per 100 g of extract, such as 0.1, 0.5, 1, 2, 3, 4, 5, or 6 mg, or any value within a range defined by any two of the aforementioned values. In some embodiments, the botanical extract includes at least 4 mg niacin per 100 g of extract, such as 1, 2, 3, 4, or 5 mg, or any value within a range defined by any two of the aforementioned values.

In some embodiments, the botanical extract has an oxygen radical absorbance capacity (ORAC) value of at least 170,000 μmol trolox equivalents (TE) per 100 g of extract, such as 190,000, 200,000, 210,000, 220,000, 230,000, 240,000, 250,000, 260,000, 270,000, 280,000, 290,000, 300,000, 310,000, 320,000, 330,000, 340,000, 350,000, 360,000, 370,000, 380,000, 390,000, 400,000, 410,000, 420,000, 430,000, 440,000, 450,000, or 460,000 μmol trolox equivalents (TE) per 100 g of extract, or any value within a range defined by any two of the aforementioned values. In some embodiments, the botanical extract has an ORAC value of at least 450,000 μmol TE per 100 g of extract.

In some embodiments, the botanical extract is prepared from botanical leaves that are 2 years or younger. In some embodiments, the botanical extract is an extract of botanical leaves that are 1 year or younger. In some embodiments, the botanical extract is prepared from botanical leaves that are 6 months or younger. In some embodiments, the botanical extract is prepared from botanical leaves that are about 3-6 months old.

In some embodiments, the botanical leaves are yerba mate leaves. In some embodiments, the botanical extract has elevated antioxidant activity. In some embodiments, the composition is formulated as a powder. In some embodiments, the composition is formulated as an antioxidant supplement. In some embodiments, the composition is formulated as a food product.

In some embodiments, the botanical extract included in the compositions disclosed herein can be prepared by any of the methods of preparing a botanical extract disclosed herein. In some embodiments, the compositions disclosed herein can be used is any of the methods of treating or improving a metabolic condition disclosed herein.

### Methods of Improving or Treating Metabolic Conditions

Some embodiments disclosed herein relate to methods of improving or treating a metabolic condition by administering to a subject in need a composition. In some embodiments, the composition includes a botanical extract. In some embodiments, the botanical extract includes at least 2.5% saponins, at least 20% polyphenols, and at least 4% xanthines. In some embodiments, the composition in formulated as a powder, tablet, capsule, or liquid.

In some embodiments, the metabolic condition is excess body weight, diabetes, insulin resistance, hyperglycemia, or any combination thereof. In some embodiments, improving or treating a metabolic condition includes reducing body weight, improving glycemic control, reducing insulin resistance, or any combination thereof. In some embodiments, the composition is configured to enhance alpha-glucosidase inhibitory effect, increase bioactivity, reduce insulin resistance, improve glycemic control, or modulate carbohydrate metabolism.

In some embodiments, the composition includes at least 1 mg of saponins, such as 1, 10, 50, 100, 200, 500, or 1000 mg, or any value within a range defined by any two of the aforementioned values. In some embodiments, the composition includes at least 600 mg of total chlorogenic acids, such as 600, 700, 800, 900, 1000, 1100, or 1200 mg of total chlorogenic acids, or any value within a range defined by any two of the aforementioned values. In some embodiments, the composition includes at least 60-180 mg of caffeine, such as 60, 70, 80, 90, 100, 110, 120, 130, 140, 150, 160, 170, or 180 mg or total caffeine, or any value within a range defined by any two of the aforementioned values.

In some embodiments, the composition is formulated as a powder, antioxidant supplement, or a food product. In some embodiments, the administering is oral ingestion. In some embodiments, the composition is administered orally at least once daily.

In some embodiments, the botanical extract used in the methods of treating or improving a metabolic condition can be prepared by any of the methods of preparing a botanical extract disclosed herein. In some embodiments, the method of treating or improving a metabolic condition can use any of the compositions disclosed herein.

### Botanical Extracts of Yerba Mate

Polyphenols are aromatic compounds characterized by hydroxylated benzene rings, classified into subfamilies based on their core structures. In yerba mate (Ilex paraguariensis), polyphenols include phenolic acids (e.g., chlorogenic acids (CQAs) and other phenolic acids), flavonoids, and saponins.

Chlorogenic acids (CQAs) are esters of caffeic acid and quinic acid. Yerba mate isomers include 3-O-caffeoylquinic acid (3-CQA), 4-O-caffeoylquinic acid (4-CQA), and 5-O-caffeoylquinic acid (5-CQA). These compounds feature a caffeoyl group (dihydroxyphenylacrylic acid) esterified to the hydroxyl groups of quinic acid (1 L-1(OH),3,4,5-tetrahydroxycyclohexanecarboxylic acid). The ortho-dihydroxy (catechol) moiety on the caffeoyl group enables potent antioxidant activity via radical scavenging and metal chelation. Yerba mate includes other phenolic acids, such as caffeic acid and ferulic acid, which are hydroxycinnamic acids with a propenoic side chain contributing to hydrophilicity and radical stabilization.

The methods of preparing a botanical extract disclosed herein preserve polyphenol integrity by using mild heating (50-70° C.) and dual aqueous phases, yielding ≥25% total polyphenols (Table 1). Younger leaves (3-6 months old) exhibit elevated CQA levels due to upregulated phenylpropanoid biosynthesis, a defense mechanism against UV stress and herbivory.

Flavonoids include rutin (quercetin-3-O-rutinoside) and quercetin derivatives, which possess a diphenylpropane (C6-C3-C6) skeleton with conjugated double bonds and hydroxyl groups at positions 3′,4′ (B-ring) for redox activity. Rutin (quercetin-3-O-rutinoside) is a flavonol glycoside comprising the aglycone quercetin linked to the disaccharide rutinose (α-L-rhamnopyranosyl-(1→6)-β-D-glucopyranose). In yerba mate, rutin accumulates in young leaves as a photoprotective agent, scavenging UV-induced reactive oxygen species (ROS). Its ortho-dihydroxy (catechol) group on the B-ring and conjugated double bond system enable potent antioxidant activity via hydrogen atom transfer (HAT) and single electron transfer (SET) mechanisms. Glycosylation of rutin enhances water solubility, facilitating extraction in aqueous phases (see, for example, Example 1), while thermal stability (≤70° C.) preserves its integrity during processing.

Saponins are amphipathic glycosides characterized by a triterpenoid or steroid aglycone (sapogenin) backbone linked to one or more sugar moieties. In yerba mate (Ilex paraguariensis), saponins are predominantly ursane-type triterpenoids, including matesaponins (e.g., matesaponin 1-5), derived from ursolic acid. These compounds exhibit a hydroxyl group at C-3 and a carboxyl group at C-28, esterified with oligosaccharide chains (e.g., β-D-glucopyranosyl, α-L-arabinofuranosyl). The structural diversity of yerba mate saponins arises from variations in glycosylation patterns (e.g., mono-, di-, or trisaccharides) and oxidation states of the sapogenin core. Saponins contribute to the bitter taste of yerba mate and demonstrate bioactivity through membrane interaction (e.g., cholesterol binding) and enzyme modulation (e.g., α-glucosidase inhibition).

The methods of preparing a botanical extract disclosed herein enrich ≥3.5% saponins (Table 1) by leveraging the hydrophilicity of glycosylated saponins in aqueous solvents. Younger leaves (3-6 months old) exhibit higher saponin content due to upregulated biosynthesis for defense against herbivores, as older leaves redirect resources toward structural lignin synthesis. Quantification via acid hydrolysis (see, for example, Example 2) confirms sapogenin profiles dominated by ursolic acid derivatives, with HPLC retention times correlating to matesaponin standards.

Xanthines are purine alkaloids derived from the methylation of xanthosine, with yerba mate including caffeine (1,3,7-trimethylxanthine) and theobromine (3,7-dimethylxanthine). Caffeine (1,3,7-trimethylxanthine) is a bicyclic compound with a purine backbone (imidazole fused to pyrimidine), methyl groups at N-1, N-3, and N-7, and a ketone at C-2 and C-6. Caffeine's planar structure facilitates adenosine receptor antagonism, promoting thermogenesis and cognitive alertness. Theobromine (3,7-dimethylxanthine) differs from caffeine by the absence of a methyl group at N-1, resulting in reduced stimulatory effects but maintained vasodilation properties.

In yerba mate, xanthines accumulate in young leaves as allelochemicals, with biosynthesis peaking at 3-6 months old (Table 1: 5.09% caffeine). The polar nature of xanthines (log P~−0.07 for caffeine) enables efficient extraction in aqueous solvents, while the disclosed two-stage process maximizes yield by disrupting leaf trichomes and parenchyma cells. Quantification via HPLC confirms xanthine stability under processing conditions (retention time: caffeine=4.1 min, λ~max~=280 nm; see, for example, Example 2).

### Numbered Embodiments

Some embodiments provided herein are described in the following numbered embodiments.
1. A composition comprising: a botanical extract, comprising: at least 2.5% saponins; at least 20% polyphenols; and at least 4% xanthines, wherein the composition is formulated as a powder, tablet, capsule, or liquid.
2. The composition of embodiment 1, wherein the botanical extract comprises at least 2.75% saponins.
3. The composition of embodiment 1 or 2, wherein the botanical extract comprises at least 3.5% saponins.
4. The composition of any one of embodiments 1-3, wherein the botanical extract comprises at least 21% polyphenols.
5. The composition of any one of embodiments 1-4, wherein the botanical extract comprises at least 25% polyphenols.
6. The composition of any one of embodiments 1-5, wherein the polyphenols comprise flavonoids, phenolic acids, stilbenes, or lignans.
7. The composition of any one of embodiments 1-6, wherein the polyphenols comprise chlorogenic acids or flavonoids.
8. The composition of embodiment 7, wherein the chlorogenic acids comprises caffeoylquinic acids, dicaffeoylquinic acids, feruloylquinic acids, cumaroylquinic acids, or a derivative thereof, or any combinations thereof.
9. The composition of embodiment 7 or 8, wherein the chlorogenic acids comprise caffeoylquinic acids.
10. The composition of any one of embodiments 1-9, wherein the botanical extract comprises at least 5% xanthines.
11. The composition of any one of embodiments 1-10, wherein the xanthines comprise aminophylline, 3-isobutyl-1-methylxanthine, paraxanthine, pentoxyifylline, diprophylline, uric acid, theophylline, caffeine, theobromine, or 7-methylxanthine, or derivatives or combinations thereof.
12. The composition of any one of embodiments 1-11, wherein the xanthines comprise caffeine.
13. The composition of any one of embodiments 1-12, wherein the botanical extract has a (1,1-diphenyl-2-picrylhydrazyl) (DPPH) IC₅₀ value of less than 1,000 μg/mL.
14. The composition of any one of embodiments 1-13, wherein the botanical extract has a (1,1-diphenyl-2-picrylhydrazyl) (DPPH) IC₅₀ value of less than 207 μg/mL.
15. The composition of any one of embodiments 1-14, wherein the botanical extract has an α-glucosidase IC₅₀ value of less than 200 g/mL.
16. The composition of any one of embodiments 1-15, wherein the botanical extract has an α-glucosidase IC₅₀ value of less than 160 g/mL
17. The composition of any one of embodiments 1-16, wherein the botanical extract further comprises one or more amino acids.
18. The composition of embodiment 17, wherein the one or more amino acids comprises histidine, isoleucine, leucine, lysine, methionine, phenylalanine, threonine, tryptophane, or valine.
19. The composition of any one of embodiments 1-18, wherein the botanical extract further comprises at least 45 mg threonine per 100 g of extract.
20. The composition of any one of embodiments 1-19, wherein the botanical extract further comprises at least 45 mg tryptophan per 100 g of extract.
21. The composition of any one of embodiments 1-20, wherein the botanical extract further comprises at least 1300 mg magnesium per 100 g of extract.
22. The composition of any one of embodiments 1-21, wherein the botanical extract further comprises at least 4 mg niacin per 100 g of extract.
23. The composition of any one of embodiments 1-22, wherein the botanical extract has an oxygen radical absorbance capacity (ORAC) value of at least 170,000 μmol trolox equivalents (TE) per 100 g of extract.
24. The composition of any one of embodiments 1-23, wherein the botanical extract has an oxygen radical absorbance capacity (ORAC) value of at least 450,000 μmol TE per 100 g of extract.
25. The composition of any one of embodiments 1-24, wherein the botanical extract is prepared from botanical leaves that are 2 years or younger.
26. The composition of any one of embodiments 1-25, wherein the botanical extract is prepared from botanical leaves that are 1 year or younger.
27. The composition of any one of embodiments 1-26, wherein the botanical extract is prepared from botanical leaves that are 6 months or younger.
28. The composition of any one of embodiments 1-27, wherein the botanical extract is prepared from botanical leaves that are about 3-6 months old.
29. The composition of any one of embodiments 1-28, wherein the botanical extract is prepared from yerba mate leaves.
30. The composition of any one of embodiments 1-29, wherein the botanical extract has elevated antioxidant activity.
31. The composition of any one of embodiments 1-30, wherein the composition is formulated as a powder.
32. The composition of any one of embodiments 1-31, wherein the composition is formulated as an antioxidant supplement.
33. The composition of any one of embodiments 1-32, wherein the composition is formulated as a food product.
34. A method of preparing a botanical extract, the method comprising: combining botanical leaves with a first solvent to form a first mixture; heating the first mixture; filtering the first mixture to obtain a first liquid extract and a first residual material; combining the first residual material with a second solvent to form a second mixture; heating the second mixture; filtering the second mixture to obtain a second liquid extract and a second residual material; and combining and concentrating the first liquid extract and second liquid extract to obtain the botanical extract.
35. The method of embodiment 34, wherein the botanical leaves are less than 6 months old.
36. The method of embodiment 34 or 35, wherein the botanical leaves are 3-6 months old.
37. The method of any one of embodiments 34-36, wherein the botanical leaves are obtained from yerba mate.
38. The method of any one of embodiments 34-37, wherein the first solvent is water.
39. The method of any one of embodiments 34-38, wherein the first solvent to botanical leaves has a ratio ranging from about 1:1 to about 20:1 in mL to grams.
40. The method of any one of embodiments 34-39, wherein the first mixture is heated to a temperature ranging from about 50° C. to about 70° C.
41. The method of any one of embodiments 34-40, wherein the first mixture is heated for a time period of about 30 minutes.
42. The method of any one of embodiments 34-41, wherein the second solvent is water.
43. The method of any one of embodiments 34-42, wherein the second solvent to first residual material has a ratio ranging from about 1:1 to about 10:1 in mL to grams.
44. The method of any one of embodiments 34-43, wherein the second mixture is heated to a temperature ranging from about 50° C. to about 70° C.
45. The method of any one of embodiments 34-44, wherein the second mixture is heated for a time period of about 30 minutes.
46. The method of any one of embodiments 34-45, further comprising spray drying the botanical extract to form a botanical extract powder.
47. The method of any one of embodiments 34-46, wherein the botanical extract comprises saponins in an amount of at least 2.5%, caffeoylquinic acids in an amount of at least 20%, and caffeine in an amount of at least 4%.
48. The method of any one of embodiments 34-47, wherein the botanical extract comprises saponins in an amount of at least 3.5%, caffeoylquinic acids in an amount of at least 25%, and caffeine in an amount of at least 5%.
49. The method of any one of embodiments 34-48, wherein the botanical extract comprises at least 45 mg threonine per 100 g of extract.
50. The method of any one of embodiments 34-49, wherein the botanical extract comprises at least 45 mg tryptophan per 100 g of extract.
51. The method of any one of embodiments 34-50, wherein the botanical extract comprises at least 1300 mg magnesium per 100 g of extract.
52. The method of any one of embodiments 34-51, wherein the botanical extract comprises at least 4 mg niacin per 100 g of extract.
53. The method of any one of embodiments 34-52, wherein the botanical extract has an oxygen radical absorbance capacity (ORAC) value of at least 450,000 μmol TE per 100 g of extract.
54. A method of improving or treating a metabolic condition, comprising administering to a subject in need a composition comprising: a botanical extract, comprising: at least 2.5% saponins; at least 20% polyphenols; and at least 4% xanthines, wherein the composition is formulated as a powder, tablet, capsule, or liquid.
55. The method of embodiment 54, wherein the metabolic condition is excess body weight, diabetes, insulin resistance, hyperglycemia, or any combination thereof.
56. The method of embodiment 54 or 55, wherein improving or treating a metabolic condition comprises reducing body weight, improving glycemic control, reducing insulin resistance, or any combination thereof.
57. The method of any one of embodiments 54-56, wherein the composition is configured to enhance alpha-glucosidase inhibitory effect, increase bioactivity, reduce insulin resistance, improve glycemic control, or modulate carbohydrate metabolism.
58. The method of any one of embodiments 54-57, wherein the composition comprises at least 1 mg of saponins.
59. The method of any one of embodiments 54-58, wherein the composition comprises at least 600 mg of total chlorogenic acids.
60. The method of any one of embodiments 54-59, wherein the composition comprises at least 60-180 mg of caffeine.
61. The method of any one of embodiments 54-60, wherein the composition is formulated as a powder, antioxidant supplement, or a food product.
62. The method of any one of embodiments 54-61, wherein the composition is formulated for oral ingestion.
63. The method of any one of embodiments 54-62, wherein the composition is administered orally at least once daily.

### EXAMPLES

Some aspects of the embodiments discussed above are disclosed in further detail in the following examples, which are not in any way intended to limit the scope of the present disclosure. Those in the art will appreciate that many other embodiments also fall within the scope of the disclosure, as it is described herein above and in the claims.

### Example 1: Yerba Mate Extraction

The yerba mate leaves used in this example were sourced from Brazil. The leaves were harvested and categorized by age based on their position along the branch (e.g., one-month-old leaves (first to third leaf pairs), two-month-old leaves (fourth to eighth leaf pairs), and six-month-old leaves (ninth to fifteenth leaf pairs), etc.). Younger leaves were harvested from apical branches. The leaves were immediately frozen to preserve freshness, then lyophilized (freeze-dried), individually crushed, and stored at −20° C. for further use in the study. This careful handling ensured the preservation of their nutrient content and quality for analysis.

The extraction of bioactive compounds from yerba mate leaves was conducted using a two-stage water extraction process. In the first stage, dried and crushed yerba mate leaves were mixed with water at a solvent-to-feed ratio of 10:1 (10 ml of water per 1 g of leaves) and heated to 55-60° C. for 30 minutes under constant agitation to facilitate the release of soluble compounds. The mixture was then filtered to separate the liquid extract from the residual plant material. The second stage involved re-extracting the residual material using a reduced solvent-to-feed ratio of 5:1 (5 mL of water per 1 g of leaves) under the same temperature and time conditions to maximize yield. The combined extracts from both stages were concentrated under vacuum at 50° C. to reduce the water content and enhance the concentration of bioactive compounds. Finally, the concentrated extract was spray-dried using a laboratory-scale spray dryer with an inlet temperature of 140° C. and an outlet temperature of 90° C. to produce a fine, free-flowing yerba mate extract powder. This powder was stored in airtight containers at −20° C. until further analysis. The two-stage extraction process, combined with vacuum concentration and spray drying, ensured efficient recovery of bioactive compounds while maintaining their stability and functionality.

### Example 2: HPLC Analysis of Bioactive Compounds

Reversed-phase high-performance liquid chromatography (RP-HPLC) was used to quantitatively and qualitatively analyze bioactive compounds in yerba mate extracts and finished products.

The analysis of various extracted compounds, including caffeine, theobromine, rutin, chlorogenic acid, and the caffeoylquinic acids (CQAs), was performed using a Thermo Vanquish Core HPLC system equipped with a binary pump, autosampler, column compartment, and diode array detector (DAD). Separation was achieved on a Thermo Scientific Accucore C18 column (2.1 mm×100 mm, 2.6 μm particle size) maintained at 35° C. The mobile phase consisted of 0.1% formic acid in water (Solvent A) and 0.1% formic acid in acetonitrile (Solvent B), with a gradient elution program starting at 95% Solvent A and 5% Solvent B, transitioning to 80% Solvent A and 20% Solvent B over 10 minutes, and returning to initial conditions by 19 minutes. The flow rate was set at 0.35 mL/min, and detection was performed at 280 nm for caffeine and theobromine, and 350 nm for rutin and CQAs.

For quantification, calibration curves were constructed using authentic reference standards (including, for example, caffeine, theobromine, rutin, chlorogenic acid, and 3,5-dicaffeoylquinic acid) prepared in serial dilutions. Theobromine was dissolved in 0.1 N sodium hydroxide, while other standards were prepared in a 70:30 methanol-water mixture. Samples were prepared by dissolving 10 mg of yerba mate extract in 5 mL of water, and filtering through a 0.22 μm membrane. 1 μL of each sample was injected into the HPLC system. Peak areas were measured, and concentrations were calculated using the external standard method, with adjustments for dilution factors.

For CQAs that lacked commercial standards, the Relative Response Factor (RRF) method was applied. Sample peak areas were compared to those of chlorogenic acid or 3,5-dicaffeoylquinic acid. This approach allowed for the estimation of 5-caffeoylquinic acid, 4-caffeoylquinic acid, 3,4-dicaffeoylquinic acid, and 4,5-dicaffeoylquinic acid, ensuring a comprehensive quantification of total CQAs. RRF method was validated against spiked samples.

This not only provided precise quantitative data but also generated a unique chromatographic “fingerprint” of yerba mate, enabling the identification of specific CQAs and other bioactive compounds. This fingerprint serves as a qualitative tool for verifying the authenticity and consistency of yerba mate products. The combination of gradient elution, dual-wavelength detection, and the RRF method ensured high sensitivity, accuracy, and reproducibility, make this HPLC technique a robust analytical tool for characterizing the bioactive chemistry of yerba mate.

To analyze the saponin content, 5 grams of dried yerba mate extract was dissolved in 15 mL of high-purity water. The solution was filtered to remove insoluble particles, and the filtrate was diluted to a final volume of 100 mL with the same solvent. Next, 15 mL of hydrochloric acid was added to achieve a concentration of 4 mL/L. The mixture was heated under reflux for 2 hours to hydrolyze the saponin molecules into sapogenins. The sapogenins were then extracted from the acidic solution using chloroform, with the extraction process repeated four times to ensure complete recovery. The combined chloroform extracts were evaporated to dryness, and the resulting residue was dissolved in acetonitrile. The solution was filtered through a 0.45-micron membrane to remove impurities, and the final filtrate was analyzed using high-performance liquid chromatography (HPLC). This entire procedure was repeated three times to ensure reproducibility.

HPLC analysis of saponins was performed using a Thermo Fisher Vanquish series HPLC system equipped with Chromeleon software, a VC-P10-A-01 pump, and a VC-D11-A-01 diode array detector (DAD) set to a detection wavelength of 203 nm. Separation was achieved using a reversed-phase Accucore C18 column (150 mm×2.1 mm, 2.6 μm particle size, Thermo Fisher). The mobile phase consisted of a mixture of acetonitrile and water (70:30, v/v), which was degassed using an ultrasonic bath and filtered under vacuum through a membrane. The flow rate was maintained at 0.4 mL/min, and the analysis was conducted at room temperature.

TABLE 1
Chemical Composition of Yerba Mate Extracts from Leaves of Different Ages

| | Extract of 3-6 | | | |
|---|---|---|---|---|
| | months old | Extract of 1 | Extract of 1.5 | Extract of 2 |
| | Yerba Mate | year old Yerba | years old Yerba | years old Yerba |
| Sample | Leaves | Mate Leaves | Mate Leaves | Mate Leaves |
| Theobromine | 0.55% | 0.68% | 0.43% | 0.57% |
| Caffeine | 5.09% | 3.85% | 2.66% | 1.52% |
| 5-CQA | 5.91% | 4.90% | 5.17% | 4.64% |
| Chlorogenic | 5.64% | 4.85% | 5.19% | 4.86% |
| acid | | | | |
| 4-CQA | 6.43% | 5.29% | 5.56% | 5.02% |
| Rutin | 0.72% | 0.52% | 0.83% | 0.69% |
| 3,4-diCQA | 2.59% | 1.25% | 1.53% | 1.14% |
| 3,5-diCQA | 1.70% | 2.45% | 1.82% | 2.36% |
| 4,5-diCQA | 3.05% | 2.98% | 2.78% | 3.43% |
| Total CQA | 25.33% | 21.71% | 22.05% | 21.45% |
| Saponin | 3.50% | 3.00% | 2.83% | 2.75% |

displays the chemical composition of yerba mate extract powders obtained from leaves of varying ages. The chemical composition of yerba mate leaves varies with age, as highlighted by the analysis of leaves at 3-6 months, 1 year, 1.5 years, and 2 years old. Theobromine shows no clear trend, with levels fluctuating from 0.55% in the youngest leaves to 0.57% in the oldest. Caffeine exhibited a distinct downward trend, decreasing from 5.09% at 3-6 months old to 1.52% at 2 years old, suggesting that younger leaves might produce more caffeine as a defense mechanism. The caffeoylquinic acids, including 5-caffeoylquinic, chlorogenic, and 4-caffeoylquinic acids, along with various dicaffeoylquinic acids, generally decreased over time. Total caffeoylquinic acids dropped from 25.33% in the youngest leaves to 21.45% in the oldest. Rutin levels did not follow a clear pattern, fluctuating between 0.52% and 0.83% across different ages. However, saponin content clearly decreased from 3.50% in 3-6-month-old leaves to 2.75% in 2-year-old leaves, indicating a reduction in defensive compounds as leaves age. This suggests that younger leaves prioritize the synthesis of protective compounds like caffeine and saponins, while older leaves might redirect resources elsewhere, possibly towards longevity or other metabolic functions. The lack of clear trends in some compounds like theobromine and rutin could point to complex regulatory mechanisms or external environmental influences.

Caffeine levels are higher in young leaves because they actively convert theobromine (a precursor) into caffeine through methylation, a process similar to that in coffee and tea. As leaves age, caffeine synthesis slows, and its degradation into theophylline and CO₂ accelerates, further reducing caffeine content. Theobromine levels peak in very young leaves but then decline as caffeine production decreases and degradation processes become more prominent.

The decline in CQAs, including chlorogenic acid and its isomers, is driven by several factors. Young leaves produce high levels of CQAs as part of their defense strategy against herbivores, pathogens, and UV radiation. However, as leaves mature, the plant shifts its focus to producing structural components like lignin and cellulose, reducing CQA synthesis. Additionally, CQAs are susceptible to oxidative degradation over time, influenced by environmental factors such as light, heat, and oxygen. Enzymes like polyphenol oxidases and peroxidases, which may become more active in older leaves, also contribute to this degradation. The biosynthesis of CQAs, which involves the esterification of quinic acid with caffeic acid, diminishes as the relevant enzymes become less active in older leaves. Reduced photosynthetic efficiency in aging leaves further shifts resources away from secondary metabolite production toward maintenance and survival. Environmental factors like nutrient availability, water stress, and light exposure, along with developmental changes such as cell wall thickening and lignin accumulation, also play a role in reducing CQA levels.

The chemical composition of leaves changes distinctly with age. There is a clear trend where caffeine levels drop sharply from 5.09% in 3-6-month-old leaves to 1.52% in 2-year-old leaves, suggesting that younger leaves produce more caffeine for defense against herbivores and pathogens, with synthesis decreasing as leaves age. Saponins also decrease steadily from 3.50% to 2.75%, reflecting a reduced need for antimicrobial protection in older leaves. Total caffeoylquinic acids (CQAs) decrease gradually, with individual CQAs like 5-CQA and 4-CQA peaking in younger leaves, while dicaffeoylquinic acids show mixed trends, indicating age-related degradation or conversion. Theobromine shows no clear age-dependent pattern, suggesting regulation by factors other than age, possibly environmental or stress-related. Rutin levels fluctuate without a discernible trend linked to leaf age, hinting at influence by external conditions like light or pathogen exposure. Overall, young leaves prioritize defensive compounds, while older leaves seem to invest less in these, possibly redirecting resources towards longevity or reproductive functions, highlighting the complex interplay of development, defense, and environment in plant metabolism.

### Example 3: DHHP Antioxidant Testing

The 2,2-diphenyl-1-picrylhydrazyl (DPPH) assay is a widely used method to evaluate the free radical scavenging activity of natural products. DPPH is a stable free radical characterized by the delocalization of its unpaired electron across the entire molecule, which prevents dimerization and gives it a deep violet color. When DPPH reacts with a compound capable of donating a hydrogen atom, it is reduced to diphenyl-picryl-hydrazine, resulting in a loss of the violet color. This color change is proportional to the antioxidant activity of the compound. In this study, the DPPH assay was performed according to the method described by (Kedare & Singh, 2011) to assess the free radical scavenging activity of yerba mate extracts.

A stock solution of each sample was prepared by dissolving 30 mg of dried yerba mate extract in 10 mL of methanol, yielding a concentration of 3 mg/mL. Serial dilutions were then prepared to obtain concentrations of 1.5, 0.75, 0.375, 0.1875, 0.09375, 0.046875, 0.0234375, and 0.01171875 mg/mL. Pure reference standards of chlorogenic acid and caffeine were used as controls and prepared in methanol at the same concentrations as the sample solutions.

A DPPH solution was prepared by dissolving 7.89 mg of DPPH in HPLC-grade methanol and diluting it to a final volume of 100 mL to achieve a concentration of 0.2 mM. This solution was further diluted to 0.1 mM for the assay. The DPPH solution was stored in the dark for 2 hours to allow the absorbance to stabilize, as it is known to decrease over time initially. The solution was kept in the dark at room temperature and used on the same day of preparation.

For the assay, 0.3 mL of each sample concentration or positive control was mixed with 2.7 mL of the 0.1 mM DPPH solution in tubes covered with aluminum foil to protect from light. The mixtures were shaken vigorously and incubated in the dark at 25° C. for 30 minutes. After incubation, the absorbance was measured at 517 nm using a microplate reader (Varioskan LUX, ThermoFisher). A control sample containing only DPPH solution and methanol (without extract) was used as a reference, and methanol served as the blank. The percentage of DPPH scavenging activity was calculated using the formula:

DPPH scavenging effect (% inhibition)={(Ac−As)/Ac)*100}
Where, Ac=absorbance of DPPH alone,
As=absorbance of DPPH along with different concentrations of extracts.

Measurements were taken at eight different concentration levels. For each concentration, the analytical sample solution was measured three times.

The IC₅₀ value, which represents the concentration of the sample required to scavenge 50% of the DPPH radicals, was determined by plotting the inhibition ratio against the sample concentrations. A regression line (y=ax+b) was fitted to the data points, and the concentration corresponding to 50% inhibition was calculated by substituting y=50 into the regression equation. The IC₅₀ value provides a quantitative measure of the antioxidant activity, with lower values indicating higher potency.

This method provides a reliable and reproducible approach to evaluate the free radical scavenging activity of yerba mate extracts, contributing to the understanding of their potential health benefits.

TABLE 2
DPPH IC₅₀ of Yerba Mate Extracts from Leaves of Different Ages

| Sample | DPPH IC₅₀, ug/ml |
|---|---|
| Caffeine | N/A |
| Chlorogenic acid | 92.17 |
| Extract of 3-6 months old Yerba Mate Leaves | 205.905 |
| Extract of 1 year old Yerba Mate Leaves | 206.428 |
| Extract of 1.5 years old Yerba Mate Leaves | 199.93 |
| Extract of 2 years old Yerba Mate Leaves | 203.463 |

The DPPH IC₅₀ value serves as a quantitative measure of antioxidant activity, with a lower value indicating a stronger capacity to scavenge free radicals. Analysis of yerba mate leaves at different ages reveals that the antioxidant activity varies mildly. The youngest leaves (3-6 months old) exhibit a DPPH IC₅₀ of 205.905 μg/mL, suggesting relatively weaker antioxidant potential. This trend continues with 1-year-old leaves at 206.428 μg/mL, showing similar activity. However, a notable peak in antioxidant activity is observed in 1.5-year-old leaves, with an IC₅₀ value of 199.930 μg/mL, indicating the relatively stronger antioxidant capacity among the samples. By 2 years, the IC₅₀ slightly increases to 203.463 μg/mL, suggesting a minor decline in antioxidant activity.

Chemically, caffeine, which is abundant in younger leaves, does not contribute to antioxidant activity as it has no DPPH scavenging ability. To contrast, chlorogenic acids (CQAs) are potent antioxidants with an IC₅₀ of around 92.17 μg/mL in their pure form. Despite high CQA content in younger leaves, their overall antioxidant activity is not as pronounced, implying that other factors play a role. Rutin, peaking at 1.5 years, could be a significant contributor to the enhanced antioxidant activity observed at this age, given its known antioxidant properties. Saponins, which decrease with age, have a minor role in antioxidant activity.

The relationship between leaf age and antioxidant function indicates that younger leaves, while rich in CQAs, do not exhibit the highest antioxidant activity due to lower rutin levels and the presence of non-antioxidant compounds like caffeine. Mid-aged leaves (1-1.5 years) demonstrate the optimal balance of CQAs and rutin, leading to the strongest antioxidant effect. As leaves age further, there's a decline in key antioxidants, resulting in slightly reduced activity. However, throughout the lifespan of yerba mate leaves, the variance in antioxidant properties remains less than 10%. This analysis highlights how leaf age critically influences the chemical composition and thus the antioxidant properties of yerba mate, which is pivotal for applications in dietary supplements or functional foods where antioxidant activity is desired. Although younger leaves do not exhibit the highest DPPH IC₅₀ value, younger leaves have the highest practical antioxidant activity, as demonstrated in Example 6.

### Example 4: α-Glucosidase Inhibitory Assay

The α-glucosidase inhibitory activity assay is a critical method for evaluating potential treatments for type 2 diabetes and related metabolic disorders. This assay measures the ability of a substance to inhibit the enzyme α-glucosidase, which is responsible for breaking down complex carbohydrates into simple sugars, primarily glucose, in the small intestine. By inhibiting α-glucosidase, carbohydrate digestion is slowed, leading to delayed and reduced glucose absorption into the bloodstream. This mechanism helps manage postprandial (after-meal) blood glucose levels, which is particularly important for individuals with type 2 diabetes or impaired glucose tolerance.

Substances with strong α-glucosidase inhibitory activity show promise as treatments for type 2 diabetes. They can help control hyperglycemia (high blood sugar) by slowing carbohydrate digestion and glucose absorption. Plant-based compounds as potential α-glucosidase inhibitors may have fewer side effects compared to synthetic drugs like acarbose, miglitol, and voglibose, which are commonly used but can cause gastrointestinal discomfort.

In this study, yerba mate extracts were evaluated for their α-glucosidase inhibitory activity, using epigallocatechin gallate (EGCG) as a positive control. EGCG, a natural compound found in green tea, is known for its α-glucosidase inhibitory properties. By comparing yerba mate extracts to EGCG, the study aimed to assess the relative effectiveness of these extracts in inhibiting α-glucosidase. This comparison helps determine whether yerba mate extracts could serve as natural alternatives for managing blood glucose levels in individuals with type 2 diabetes or related metabolic disorders.

The α-glucosidase inhibitory activity of the extracts and fractions was evaluated using a standard method with minor modifications. The procedure was conducted in a 96-well plate, where the reaction mixture consisted of 50 μL of phosphate buffer (100 mM, pH 6.8), 10 μL of α-glucosidase solution (1 U/mL), and 20 μL of the extract or fraction at varying concentrations (0.1, 0.2, 0.3, 0.4, and 0.5 mg/mL). The mixture was preincubated at 37° C. for 15 minutes, after which 20 μL of p-nitrophenyl-α-D-glucopyranoside (p-NPG, 5 mM) was added as a substrate. The mixture was then incubated further at 37° C. for 20 minutes. To stop the reaction, 50 μL of sodium carbonate (Na₂CO₃, 0.1 M) was added. The absorbance of the released p-nitrophenol was measured at 405 nm using a microplate reader. Acarbose, a known α-glucosidase inhibitor, was used as a standard at concentrations ranging from 0.1 to 0.5 mg/mL, and a control without the test substance was included for comparison. Each experiment was performed in triplicate to ensure accuracy. The results were expressed as percentage inhibition, calculated using the formula:

$$Inhibitory activity ( \% ) = ( 1 - As / Ac ) \times 100$$

Where As is the absorbance in the presence of test substance and Ac is the absorbance of control.

TABLE 3
α-Glucosidase Inhibitory Activity of Yerba
Mate Extracts from Leaves of Different Ages

| | a-Glucosidase inhibition |
|---|---|
| Sample | assay, IC₅₀, ug/ml |
| Caffeine | N/A |
| EGCG | 99 |
| Chlorogenic acid | 190 |
| Extract of 3-6 months old Yerba Mate Leaves | 157.6 |
| Extract of 1 year old Yerba Mate Leaves | 200.0 |
| Extract of 1.5 years old Yerba Mate Leaves | 195.1 |
| Extract of 2 years old Yerba Mate Leaves | 199.4 |

The analysis of yerba mate leaf extracts revealed a significant correlation between leaf age, chemical composition, and α-glucosidase inhibitory activity, as quantified by IC₅₀ values, where a lower value indicates stronger inhibition. Young leaves, aged 3-6 months old, demonstrate the most potent inhibition with an IC₅₀ of 157.6 μg/mL, which diminishes to 200.0 μg/mL in 1-year-old leaves, 195.1 μg/mL in 1.5-year-old leaves, and 199.4 μg/mL in 2-year-old leaves. This trend suggests that α-glucosidase inhibitory strength decreases as leaves age. Young leaves contain the highest levels of saponins (3.50%) and exhibit the strongest inhibition, while older leaves show a decline in saponin content to 2.75% and a corresponding increase in IC₅₀ values, indicating weaker inhibition. Although chlorogenic acids (CQAs) are known for their inhibitory effects, their gradual decrease from 25.33% in young leaves to 21.45% in older ones does not fully account for the observed changes in IC₅₀ values. The levels of caffeine and rutin do not correlate consistently with the inhibition, underscoring the pivotal role of saponins. Pure chlorogenic acid has an IC₅₀ of approximately 190 μg/mL, aligning more with the activity of mid-aged or older leaves rather than the young ones. The robust inhibition in young leaves suggests that saponins might play a synergistic or even primary role in enhancing α-glucosidase inhibition beyond what CQAs alone can achieve.

The health benefits of yerba mate, including its α-glucosidase inhibition, are influenced by a variety of bioactive compounds such as polyphenols (including chlorogenic acids, caffeic acid, and quercetin), flavonoids, saponins, and methylxanthines, like caffeine and theobromine. Polyphenols and flavonoids act as potent antioxidants and inhibit α-glucosidase by binding to its active site, preventing the breakdown of complex carbohydrates into glucose, thus slowing its absorption. Saponins, on the other hand, interact with the enzyme's structure, altering its conformation and reducing its activity. Although caffeine and theobromine are not direct inhibitors, they might enhance the overall bioactivity through metabolic modulation or synergy with other compounds.

The concentration of these bioactive compounds varies with leaf age. Younger leaves have higher concentrations due to active synthesis for defense and metabolic functions, leading to stronger α-glucosidase inhibition. Conversely, as leaves age, these compounds degrade or are metabolized, reducing their concentration and the leaf's inhibitory potential. The mechanism of inhibition involves competitive inhibition where compounds like chlorogenic acids and flavonoids compete with natural substrates for the enzyme's active site, and non-competitive inhibition, where binding at allosteric sites changes the enzyme's shape, reducing its efficiency. Synergistic effects among the compounds further enhance inhibition.

The health benefits of this inhibition include improved glycemic control, potentially reducing insulin resistance, providing antioxidant effects, and aiding in weight management by slowing carbohydrate digestion. The variability in the efficacy of yerba mate for α-glucosidase inhibition is thus directly related to leaf age and the resultant changes in chemical composition. In conclusion, younger yerba mate leaves, with their richer content of bioactive compounds, offer superior α-glucosidase inhibition, which is crucial for therapeutic applications like diabetes management, emphasizing the importance of leaf age in optimizing health benefits.

### Example 5: Nutritional Profile of Yerba Mate Extract from Young Leaves

This example demonstrates the nutritional analysis of a yerba mate extract derived from young leaves (3-6 months old), and compares it to literature data on yerba mate extracts to demonstrate its superior nutritional value. The extract was subjected to comprehensive testing for amino acids, minerals, and water-soluble B vitamins to evaluate its potential as a nutrient-rich dietary supplement or functional food ingredient. Analytical testing was conducted by Eurofins Food Testing Singapore Pte. Ltd. employing validated methods: High-Performance Liquid Chromatography (HPLC) for amino acids (TAALC_S), Inductively Coupled Plasma-Optical Emission Spectrometry (ICP-OES) for minerals (ICP_S), and Liquid Chromatography-Mass Spectrometry (LC-MS/MS) for B vitamins (BLCMS_S).

The amino acid profile was determined using automated pre-column derivatization and HPLC, as per Schuster (1988) (R. Schuster, 1988), with results expressed in mg/100 g of extract powder (Table 4). The extract contains all nine essential amino acids (EAAs)—histidine, isoleucine, leucine, lysine, methionine, phenylalanine, threonine, tryptophan, and valine—required for human nutrition, alongside several non-essential amino acids.

TABLE 4
Amino Acid Composition of Yerba Mate
Extract Compared to Literature Data

| | | | |
|---|---|---|---|
| | Sample | Literature | |
| | Range | Range | Percentage Change from |
| Amino Acid | (mg/100 g) | (mg/100 g)* | Literature Values |

Essential Amino Acids

| | | | |
|---|---|---|---|
| Histidine | 20.7 | 5-15 | 38-314% higher |
| Isoleucine | 30.5 | 10-25 | 22-205% higher |
| Leucine | 47.5 | 15-35 | 36-216% higher |
| Lysine | 56.9 | 20-40 | 42-184% higher |
| Methionine | 13.5 | 5-10 | 35-170% higher |
| Phenylalanine | 32.6 | 10-25 | 30-226% higher |
| Threonine | 45.6 | 10-20 | 128-356% higher |
| Tryptophan | 45 | 5-20 | 125-800% higher |
| Valine | 47.2 | 15-30 | 57-214% higher |

Non-Essential Amino Acids

| | | | |
|---|---|---|---|
| Alanine | 60 | 20-50 | 20-200% higher |
| Arginine | 245 | 50-150 | 63-390% higher |
| Cystine | 35 | 10-25 | 40-250% higher |
| Glutamic Acid | 420 | 100-300 | 40-320% higher |
| Glycine | 103 | 30-80 | 29-243% higher |
| Proline | 48.3 | 15-40 | 21-222% higher |
| Serine | 54.5 | 20-45 | 21-172% higher |
| Tyrosine | 30.8 | 10-25 | 23-208% higher |

*Literature ranges approximated from studies on yerba mate extracts (e.g., Heck & De Mejia, 2007; Burris et al., 2012)(Burris, Harte, Davidson, et al., 2012; Heck & De Mejia, 2007b), typically from leaves of varying or unspecified ages.

The yerba mate extract from young leaves exceeds literature ranges for all EAAs, with threonine (45.6 mg/100 g) showing a 128-356% increase over the reported range (10-20 mg/100 g), and tryptophan (45.0 mg/100 g) up to 800% higher than the maximum (20 mg/100 g). Non-essential amino acids, such as arginine (245 mg/100 g) and glutamic acid (420 mg/100 g), are also 63-390% and 40-320% higher, respectively. This enrichment likely stems from the selection of young leaves, which are metabolically active and prioritize protein synthesis for growth and defense, unlike older leaves where protein degradation and resource reallocation predominate.

Mineral analysis was conducted using ICP-OES (AOAC Methods 984.27, 985.01, 2011.14, modified), quantifying key macro- and microminerals essential for human health (Table 5). The results highlight the extract's exceptional mineral potency.

TABLE 5
Mineral Composition of Yerba Mate
Extract Compared to Literature Data

| | Sample | Literature | |
|---|---|---|---|
| | Range | Range | Percentage Change from |
| Mineral | (mg/100 g) | (mg/100 g)* | Literature Values |
| Calcium | 278 | 50-150 | 85-456% higher |
| Copper | 0.563 | 0.1-0.5 | 13-463% higher |
| Iron | 1.64 | 0.5-2.0 | Within range, near upper limit |
| Magnesium | 1370 | 30-100 | 1270-4467% higher |
| Manganese | 225 | 10-50 | 350-2150% higher |
| Phosphorus | 145 | 50-150 | Within range, near upper limit |
| Potassium | 3530 | 100-300 | 1077-3430% higher |
| Sodium | 25.6 | 5-20 | 28-412% higher |
| Zinc | 9.08 | 0.2-1.0 | 808-4440% higher |

*Literature ranges sourced from Burris et al. (2012), Heck & De Mejia (2007), and Rzasa-Duran et al. (2022)(Burris et al., n.d.; Heck & De Mejia, 2007b; Rzasa-Duran et al., 2022b) for yerba mate extracts from mixed-age leaves.

The yerba mate extract demonstrates significantly higher mineral content compared to literature values, particularly for magnesium (1370 mg/100 g, 1270-4467% higher than 30-100 mg/100 g), potassium (3530 mg/100 g, 1077-3430% higher than 100-300 mg/100 g), manganese (225 mg/100 g, 350-2150% higher than 10-50 mg/100 g), and zinc (9.08 mg/100 g, 808-4440% higher than 0.2-1.0 mg/100 g). Calcium (278 mg/100 g) and sodium (25.6 mg/100 g) also exceed typical ranges by 85-456% and 28-412%, respectively. Iron (1.64 mg/100 g) and phosphorus (145 mg/100 g) fall within literature ranges but align with the upper limits, indicating robust accumulation. This mineral richness is attributable to the young leaves' active nutrient uptake, facilitated by nutrient-rich compost and sustainable cultivation practices, contrasting with older leaves where mineral levels decline due to metabolic shifts.

Water-soluble B vitamins were analyzed using LC-MS/MS (internally developed method), highlighting their role in energy metabolism and neurological health (Table 6). The yerba mate extract's B-vitamin profile was quantified, with results compared to literature data.

TABLE 6
B-Vitamin Content of Yerba Mate Extract
Compared to Literature Data

| | | Literature | |
|---|---|---|---|
| | Sample | Range | Change Compared to |
| Vitamin | (mg/100 g) | (mg/100 g)* | Literature Values |
| Vitamin B1 | 0.196 | 0.1-0.5 | Within range, near |
| (Thiamine) | | | midpoint |
| Vitamin B2 | 0.654 | 0.2-0.8 | Within range, near |
| (Riboflavin) | | | upper limit |
| Vitamin B3 | 4.03 | 1.0-5.0 | Within range, near |
| (Niacin) | | | upper limit |
| Vitamin B5 | 0.422 | 0.5-2.0 | Below range, slightly |
| (Pantothenic Acid) | | | lower |
| Vitamin B6 | 0.0513 | 0.1-0.6 | Below range, notably |
| (Pyridoxine) | | | lower |
| Vitamin B7 | <0.00261 | 0.001-0.01 | Below range, near |
| (Biotin)** | | | detection limit |

*Literature ranges derived from Heck & De Mejia (2007) (Heck & De Mejia, 2007c)and Rzasa-Duran et al. (2022) (Rzasa-Duran et al., 2022b) for yerba mate extracts from mixed-age leaves.
**Biotin reported as <2.61 mcg/100 g (i.e., <0.00261 mg/100 g), indicating it is below the method's quantification limit; literature range approximated from plant-based sources.

The yerba mate extract's B-vitamin profile shows a mixed comparison to literature data. Niacin (4.03 mg/100 g) and riboflavin (0.654 mg/100 g) are within the upper limits of reported ranges (1.0-5.0 mg/100 g and 0.2-0.8 mg/100 g, respectively), reflecting robust synthesis in young leaves tied to methylxanthine metabolism (e.g., caffeine at 5.09%). Thiamine (0.196 mg/100 g) falls within the mid-range (0.1-0.5 mg/100 g), indicating moderate enrichment. However, pantothenic acid (0.422 mg/100 g) and pyridoxine (0.0513 mg/100 g) are below their respective ranges (0.5-2.0 mg/100 g and 0.1-0.6 mg/100 g), suggesting lower accumulation or degradation during processing. While pantothenic acid and pyridoxine levels are modest, the overall nutritional profile exceeds the literature norms due to amino acids and minerals. Biotin (<0.00261 mg/100 g) is near the detection limit and below typical plant-based ranges (0.001-0.01 mg/100 g), possibly due to minimal biosynthetic priority in young leaves. Despite these variations, the overall nutritional profile remains strong, with other B vitamins supporting energy metabolism and cognitive health.

The Yerba Mate Extract from young leaves (3-6 months old) described in this example exhibits a superior nutritional profile compared to literature data on yerba mate extracts from mixed or older leaves, delivering a potent combination of essential amino acids (EAAs), minerals, and B vitamins with significant health benefits. Its complete EAA profile, including threonine at 45.6 mg/100 g (128-356% higher than the 10-20 mg/100 g literature range), positions it as a valuable plant-based protein source. Threonine supports mucin synthesis in the gut, enhancing intestinal barrier function and immune defense (Wu, 2009), while tryptophan (45.0 mg/100 g, 125-800% higher) serves as a precursor to serotonin and melatonin, promoting mood stability, sleep regulation, and neuroprotection (Fernstrom, 2012). Other EAAs, such as leucine (47.5 mg/100 g, 36-216% higher) and lysine (56.9 mg/100 g, 42-184% higher), stimulate muscle protein synthesis via the mTOR pathway and support collagen formation for connective tissue health, respectively (Paddon-Jones et al., 2004). This robust amino acid composition underpins muscle repair, immune function, and neurotransmitter synthesis, offering a dietary alternative to animal-derived proteins.

The mineral content further amplifies the extract's therapeutic potential, with magnesium (1370 mg/100 g, 1270-4467% higher than 30-100 mg/100 g), potassium (3530 mg/100 g, 1077-3430% higher than 100-300 mg/100 g), manganese (225 mg/100 g, 350-2150% higher than 10-50 mg/100 g), and zinc (9.08 mg/100 g, 808-4440% higher than 0.2-1.0 mg/100 g) far exceeding literature ranges. Magnesium acts as a cofactor in over 300 enzymatic reactions, including ATP production and glucose metabolism, supporting energy homeostasis and reducing muscle cramps (Al Alawi et al., 2018; Fatima et al., 2024). Potassium regulates fluid balance and nerve signaling, contributing to cardiovascular health by modulating blood pressure via sodium-potassium pump activity (Haddy et al., 2006; B. S. Kim et al., 2024; Terker et al., 2015). Manganese, a component of manganese superoxide dismutase (MnSOD), enhances antioxidant defenses against oxidative stress, protecting mitochondrial function and reducing inflammation (Holley et al., 2011; Li & Yang, 2018). Zinc bolsters immune function by supporting T-cell development and cytokine production, while also aiding wound healing and DNA synthesis (Skrajnowska & Bobrowska-Korczak, 2019; Wessels et al., 2017). Calcium (278 mg/100 g, 85-456% higher) strengthens bone mineralization, complementing magnesium for skeletal integrity. These mineral levels enhance bone health, muscle function, immune support, and metabolic resilience, positioning the extract as a multi-faceted nutritional powerhouse.

The B-vitamin profile, with niacin (4.03 mg/100 g) and riboflavin (0.654 mg/100 g) near the upper limits of literature ranges (1.0-5.0 mg/100 g and 0.2-0.8 mg/100 g), contributes significantly to metabolic and neurological health. Niacin, as a precursor to NAD+, supports cellular energy production and DNA repair, offering potential benefits for lipid metabolism and cardiovascular risk reduction (Amjad et al., 2021; Pirinen et al., 2020). Riboflavin, a component of FAD and FMN coenzymes, facilitates redox reactions in the electron transport chain, enhancing energy metabolism and protecting against oxidative damage (Aragão et al., 2024). Thiamine (0.196 mg/100 g, within 0.1-0.5 mg/100 g) supports carbohydrate metabolism as part of thiamine pyrophosphate (TPP), critical for nerve function and preventing beriberi-like fatigue (Mrowicka et al., 2023). However, pantothenic acid (0.422 mg/100 g, below 0.5-2.0 mg/100 g) and pyridoxine (0.0513 mg/100 g, below 0.1-0.6 mg/100 g) are lower than typical ranges, possibly due to heat-induced degradation during processing or reduced biosynthesis in young leaves focused on growth rather than coenzyme storage. Pantothenic acid (as Coenzyme A) and pyridoxine (as PLP) are essential for fatty acid metabolism and amino acid transamination, respectively, suggesting that while present, their lower levels may limit certain metabolic contributions compared to literature norms. Biotin (<0.00261 mg/100 g) is negligible, consistent with its minor role in plant tissues.

This nutritional enrichment is attributable to the young leaves, harvested at peak metabolic activity. Young leaves prioritize the synthesis of proteins, minerals, and bioactives for growth and defense, as evidenced by the high saponin (3.50%), chlorogenic acid (CQA, 25.33%), and caffeine (5.09%) levels in Example 1. Saponins enhance glycemic control by inhibiting carbohydrate digestion enzymes, while chlorogenic acids provide potent antioxidant and anti-inflammatory effects via Nrf2 pathway activation, complementing the mineral-driven antioxidant capacity. Caffeine boosts thermogenesis and cognitive performance by antagonizing adenosine receptors. In contrast, literature data typically reflect extracts from leaves of varying maturities, where nutrient degradation (e.g., protein hydrolysis, mineral leaching) and metabolic shifts toward lignification reduce amino acid, mineral, and vitamin concentrations. The yerba mate extract's superiority-confirmed by nutrition testing data across amino acids, minerals, and B vitamins-underscores the advantage of the disclosed methods, delivering a product with enhanced therapeutic and dietary potential. These benefits include improved glycemic regulation, antioxidant protection, muscle and bone health, immune modulation, and cognitive enhancement, positioning it as a superior functional ingredient for health optimization.

### Example 6: ORAC Antioxidant Assay

The Oxygen Radical Absorbance Capacity (ORAC) assay was utilized to evaluate the antioxidant capacity of the Yerba Mate Extract derived from leaves of varying ages. This assay measures the ability of the extract to neutralize peroxyl radicals, a biologically relevant reactive oxygen species (ROS), by monitoring the fluorescence decay of fluorescein over time, following an adapted protocol from (Prior et al., 2003) with modifications tailored for plant extract analysis to ensure reproducibility. The materials and reagents included yerba mate extract powders from four lots—3-6 months, 1 year, 1.5 years, and 2 years—prepared as 100% powder via water extraction and spray-drying, dissolved in 50 mM phosphate buffer (pH 7.4) prepared by dissolving 6.8 g of monobasic potassium phosphate (KH₂PO₄) and 8.7 g of dibasic potassium phosphate (K₂HPO₄) in 1 L of deionized water, adjusted to pH 7.4 with 1 M NaOH or HCl. Additional reagents comprised fluorescein sodium salt (120 nM stock solution), 2,2′-azobis(2-amidinopropane) dihydrochloride (AAPH, 40 mM fresh solution), and Trolox (100 μM stock solution with serial dilutions of 6.25, 12.5, 25, 50, 75 μM) as the standard, with equipment including a microplate reader (excitation 485 nm, emission 520 nm), 96-well black microplates, and an incubator set at 37° C. Sample preparation involved weighing 0.1 g of each extract powder, dissolving it in 10 mL of phosphate buffer to achieve a 10 mg/ml concentration, vortexing for 2 minutes, sonicating for 10 minutes at 25° C., filtering through a 0.45 μm PVDF syringe filter, and diluting 1:100 to 100 μg/mL for the assay, with storage at 4° C. and use within 2 hours to prevent degradation. The assay procedure entailed adding 120 μL of 78 nM fluorescein working solution to each well of a 96-well black microplate, followed by 20 μL of diluted sample, Trolox standards, or blank (phosphate buffer) in triplicate, incubating at 37° C. for 15 minutes, then adding 60 μL of 40 mM AAPH to initiate the reaction, with fluorescence measured every 2 minutes for 90 minutes at 37° C. with gentle shaking before each reading. Data analysis involved calculating the area under the curve (AUC) using the trapezoidal method (AUC=1+Σ[(Fᵢ+Fᵢ₊₁)/2×(tᵢ₊₁−tᵢ)]), subtracting the blank AUC to obtain net AUC, generating a linear calibration curve from Trolox standards (R²≥0.99), and determining the ORAC value in micromoles of Trolox equivalents per 100 g (μmol TE/100 g) using the formula: ORAC=(TE concentration (μM)/Sample concentration (μg/mL))×Dilution factor×10⁵, with triplicate analysis ensuring a coefficient of variation (CV)<5%. Quality control measures included verifying the Trolox calibration linearity (R²≥0.99), using a 50 μM Trolox positive control per plate, monitoring blank fluorescence decay (>90% within 90 minutes), and protecting light-sensitive reagents by storing them at 4° C.

This example evaluates the antioxidant capacity of the Yerba Mate Extract derived from young leaves (3-6 months old) and compares it to extracts from leaves of varying ages, using the Oxygen Radical Absorbance Capacity (ORAC) assay.

The ORAC assay provided additional information to the DPPH method, addressing the observation that DPPH results for these extracts were not significantly different across leaf ages. While DPPH measures free radical scavenging via a single electron transfer reaction, it relies on a stable synthetic radical that does not fully mimic the biological oxidative stress conditions encountered in vivo. In contrast, ORAC employs a biologically relevant peroxyl radical system, generated by AAPH, which simulates lipid peroxidation processes that damage cell membranes and contribute to chronic diseases (Huang et al., 2005; Karadag et al., 2009). Therefore, ORAC provides additional information for evaluating the extract's potential to mitigate oxidative damage in physiological contexts.

TABLE 7
ORAC Values of Yerba Mate Extract Across Leaf Ages

| | ORAC (μmol |
|---|---|
| Sample | TE/100 g) |
| Extract of 3-6 months old Yerba Mate Leaves | 456,668 |
| Extract of 1 year old Yerba Mate Leaves | 268,884 |
| Extract of 1.5 years old Yerba Mate Leaves | 280,940 |
| Extract of 2 years old Yerba Mate Leaves | 198,645 |

The extract from young leaves (3-6 months old) exhibited an ORAC value of 456,668 μmol TE/100 g, significantly higher than extracts from older leaves. The 1-year extract showed a 41% reduction (268,884 μmol TE/100 g), while the 1.5-year and 2-year extracts declined further to 280,940 μmol TE/100 g (38% reduction) and 198,645 μmol TE/100 g (56% reduction), respectively, relative to the 3-6 months old sample. This trend of a peak at 3-6 months followed by a decline with age is explained by the metabolic dynamics of yerba mate leaves. Young leaves prioritize the synthesis of bioactive antioxidants, such as chlorogenic acids (CQAs, 25.33%) and saponins (3.50%) reported in Example 1, to protect against environmental stressors during growth. CQAs scavenge free radicals and upregulate endogenous antioxidants via the Nrf2 pathway, while saponins enhance cellular resilience by modulating oxidative stress responses. Caffeine (5.09%) also contributes to ROS scavenging and mitochondrial protection. As leaves mature, metabolic activity shifts toward lignification and resource reallocation, reducing the accumulation of these soluble antioxidants and leading to a corresponding decline in ORAC values.

To contextualize the Yerba Mate Extract's antioxidant capacity, its best ORAC value (456,668 μmol TE/100 g from 3-6 months old leaves) is compared with those of other potent natural antioxidants, measured on a per 100 g basis where available. Green tea, a widely recognized antioxidant source, typically ranges from 1,253 to 62,000 μmol TE/100 g depending on preparation and source, with an average often cited around 125,000 μmol TE/100 g. Rosemary, rich in carnosic acid and rosmarinic acid, exhibits ORAC values of approximately 165,280 μmol TE/100 g, reflecting its potent scavenging activity. Other notable antioxidants include guayusa (up to 127,000 μmol TE/100 g), grape seed flour (100,000 μmol TE/100 g), raw cocoa powder (80,933 μmol TE/100 g), turmeric (127,068 μmol TE/100 g), cloves (314,446 μmol TE/100 g), cinnamon (131,420 μmol TE/100 g), oregano (175,295 μmol TE/100 g), and dark chocolate (20,823 μmol TE/100 g). Fruits and berries such as wild blueberries (9,621 μmol TE/100 g), cranberries (9,584 μmol TE/100 g), and goji berries (25,000 μmol TE/100 g) also contribute, while vegetables like kale (1,770 μmol TE/100 g) and broccoli (3,083 μmol TE/100 g) offer lower but significant values.

The yerba mate extract's ORAC value of 456,668 μmol TE/100 g surpasses all these natural antioxidants, exceeding green tea by 3.6 to 365 times, rosemary by 2.8 times, guayusa by 3.6 times, and even cloves (the highest among spices) by 1.5 times. This exceptional potency is likely due to the concentrated levels of CQAs (25.33%), saponins (3.50%), and caffeine (5.09%) in the young leaf extract, amplified by the disclosed methods for preparing a botanical extract. Variability in reported ORAC values for other sources-often influenced by preparation methods, drying conditions, or sample concentration-suggests caution in direct comparisons, but the current yerba mate extract's consistency across triplicate testing reinforces its superiority.

Notably, the ORAC value of the 3-6 months old leaf extract (456,668 μmol TE/100 g) is significantly higher than that of chlorogenic acid alone, which ranges from 62,000 to 67,000 μmol TE/100 g, highlighting the extract's superior antioxidant capacity despite its high CQA content (25.33%), likely due to the synergistic contributions of saponins (3.50%), caffeine (5.09%), and other phytochemicals present in young leaves.

Oxidative stress, characterized by an imbalance between ROS production and antioxidant defenses, is a central mechanism in aging, neurodegenerative diseases, and metabolic disorders (Singh et al., 2019). Peroxyl radicals, generated during lipid peroxidation, damage cell membranes, trigger inflammation, and contribute to atherosclerosis, insulin resistance, and neuronal degradation (Ayala et al., 2014; Chandimali et al., 2025). The ORAC assay's focus on peroxyl radical neutralization provides a direct measure of the extract's capacity to protect against these processes. The yerba mate extract's ORAC value of 456,668 μmol TE/100 g for the 3-6 months old leaves indicates exceptional antioxidant potency, surpassing typical values for plant extracts (e.g., green tea: ~125,000 μmol TE/100 g). Further, the yerba mate extract's ORAC value of 456,668 μmol TE/100 g for the 3-6 months old leaves surpassed the ORAC values of 1 year old, 1.5 years old, and 2 year old yerba mate leaves. This highlights its superiority in combating oxidative damage at concentrations relevant to dietary supplementation, offering potential protection against oxidative stress-related conditions.

The high ORAC value of the yerba mate extract from young leaves underscores its therapeutic potential for mitigating oxidative stress, with significant implications for health. By neutralizing peroxyl radicals, the extract can reduce lipid peroxidation, a key contributor to cardiovascular disease, by protecting endothelial cells and lowering oxidized LDL levels. This antioxidant capacity also supports glycemic control by preserving pancreatic beta-cell function and improving insulin sensitivity, which are often impaired by ROS in diabetes. Furthermore, the extract's ability to combat oxidative stress offers neuroprotective benefits, potentially slowing age-related cognitive decline and reducing the risk of neurodegenerative diseases like Alzheimer's, where oxidative damage plays a pivotal role. These health benefits align with the extract's therapeutic potential, positioning it as a valuable functional ingredient for antioxidant supplementation, cardiovascular health, glycemic regulation, and anti-aging applications.

The decline in ORAC values with leaf age underscores the advantages of the disclosed methods for preparing a botanical extract. The disclosed methods maximize the accumulation of polyphenolic antioxidants and other bioactives, as evidenced by the extract's high CQA, saponin, and caffeine content. In contrast, literature data on older leaf extracts reflect lower antioxidant capacities due to degradation of these compounds during maturation and processing. The yerba mate extract's superior ORAC profile, validated by this assay, enhances its therapeutic potential for mitigating oxidative stress, supporting cardiovascular health, improving glycemic control, and combating age-related decline, thereby reinforcing the disclosed methods' improved high-potency antioxidant botanical extract.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise. While several examples have been described in detail, it is to be understood that the disclosed examples may be modified. Therefore, the foregoing description is to be considered non-limiting.

Features, materials, characteristics, or groups described in conjunction with a particular aspect, or example are to be understood to be applicable to any other aspect or example described in this section or elsewhere in this specification unless incompatible therewith. All of the features disclosed in this specification (including any accompanying claims or abstract), and/or all of the steps of any method or process so disclosed, may be combined in any combination, except combinations where at least some of such features and/or steps are mutually exclusive. The protection is not restricted to the details of any foregoing examples. The protection extends to any one, or any combination, of the features disclosed in this specification (including any accompanying claims or abstract), or to any one, or any combination, of the steps of any method or process so disclosed.

Furthermore, certain features that are described in this disclosure in the context of separate implementations may also be combined in a single implementation. Additionally, various features described in the context of a single implementation may be implemented in multiple implementations separately or in a suitable sub-combination. Moreover, although features may be described above as acting in certain combinations, one or more feature from a claimed combination may be excised from the combination, and the combination may be claimed as a sub-combination or variation of a sub-combination.

For purposes of this disclosure, certain aspects, advantages, and features are described herein. Not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without user input or prompting, whether these features, elements, and/or steps are included or are to be performed in any particular example.

The scope of the present disclosure is not intended to be limited by the specific disclosures of preferred examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive. Although certain dependent claims may be described as depending from a single preceding claim, it is to be understood that each such dependent claim may alternatively be construed as depending from any one of the preceding claims, whether independent or dependent, unless expressly stated otherwise. All such alternative dependencies and combinations are expressly contemplated as part of the disclosure.

The described embodiments and examples of the present disclosure are intended to be illustrative rather than restrictive, and are not intended to represent every embodiment or example of the present disclosure, and thus, are not to be limited in scope by the specific embodiments and examples described herein. While the fundamental features of the disclosure as applied to various specific embodiments thereof have been shown, described, and pointed out, it will also be understood that various omissions, substitutions, and changes in the details of the compositions and methods that are disclosed, may become apparent and may be made by those skilled in the art without departing from the spirit of the disclosure. For example, it is expressly intended that all combinations of those method steps that perform substantially the same function in substantially the same way to achieve the same results are within the scope of the disclosure. Moreover, it should be recognized that method steps shown and/or described in connection with any disclosed form or embodiment of the disclosure may be incorporated in any other disclosed or described or suggested form or embodiment as a general matter of design choice. Further, various modifications and variations may be made without departing from the spirit or scope of the disclosure as set forth in the following claims both literally and in equivalents recognized in law.

Unless defined otherwise, technical and scientific terms used herein have the same meaning as commonly understood by one of ordinary skill in the art to which the present disclosure belongs. For purposes of the present disclosure, the following terms are defined below.

All publications, patent applications, patents, and other references mentioned herein are incorporated by reference in their entirety. In case of conflict, the present specification, including explanations of terms, will control. In addition, the materials, methods, and examples are illustrative only and not intended to be limiting.

As used herein, the term “about” or “approximately” in reference to a numerical value for a particular parameter is inclusive of the numerical value and a degree of variance from the numerical value that one of ordinary skill in the art would understand is within acceptable tolerances for the particular parameter. For example, “about” or “approximately” in reference to a numerical value may include additional numerical values within a range of from 80.0 percent to 120.0 percent of the numerical value, such as within a range of from 90.0 percent to 110.0 percent of the numerical value, within a range of from 95.0 percent to 105.0 percent of the numerical value, within a range of from 97.5 percent to 102.5 percent of the numerical value, within a range of from 99.0 percent to 101.0 percent of the numerical value, within a range of from 99.5 percent to 100.5 percent of the numerical value, or within a range of from 99.9 percent to 100.1 percent of the numerical value.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those of skill within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description or claims, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

Any of the features of an embodiment of the first through second aspects is applicable to all aspects and embodiments identified herein. Moreover, any of the features of an embodiment of the first through third aspects is independently combinable, partly or wholly with other embodiments described herein in any way, e.g., one, two, or three or more embodiments may be combinable in whole or in part. Further, any of the features of an embodiment of the first through third aspects may be made optional to other aspects or embodiments.

As used herein, the term “composition” has its ordinary meaning as commonly understood by those skilled in the art, and includes a material or compound composed of two or more different substances, or a product containing two or more substances, whether resulting from chemical union or from mechanical mixture of the substances, and whether the substances are gases, fluids, powders or solids.

As used herein, the term “obtained” has its ordinary meaning as commonly understood by those skilled in the art, and includes originating from, being generated from, and being synthesized from a designated source(s) by known purification, separation, and/or extraction techniques.

As used herein, the term “combination thereof” has its ordinary meaning as commonly understood by those skilled in the art and includes a mixture, a blend, a stack, a reaction product, or any combination, whether singular or plural, of two or more constituting elements.

What is claimed is:

1. A composition comprising:
a botanical extract, comprising:
at least 2.5% saponins;
at least 20% polyphenols; and
at least 4% xanthines,
wherein the composition is formulated as a powder, tablet, capsule, or liquid.

2. The composition of claim 1, wherein the botanical extract comprises at least 2.75% saponins.

3. The composition of claim 1, wherein the botanical extract comprises at least 3.5% saponins.

4. The composition of claim 1, wherein the botanical extract comprises at least 21% polyphenols.

5. The composition of claim 1, wherein the botanical extract comprises at least 25% polyphenols.

6. The composition of claim 1, wherein the polyphenols comprise chlorogenic acids or flavonoids.

7. The composition of claim 6, wherein the chlorogenic acids comprise caffeoylquinic acids.

8. The composition of claim 1, wherein the botanical extract comprises at least 5% xanthines.

9. The composition of claim 1, wherein the xanthines comprise caffeine.

10. The composition of claim 1, wherein the botanical extract has a (1,1-diphenyl-2-picrylhydrazyl) (DPPH) IC₅₀ value of less than 1,000 μg/mL.

11. The composition of claim 1, wherein the botanical extract has an α-glucosidase IC₅₀ value of less than 200 μg/mL.

12. The composition of claim 1, wherein the botanical extract has an oxygen radical absorbance capacity (ORAC) value of at least 170,000 μmol trolox equivalents (TE) per 100 g of extract.

13. The composition of claim 1, wherein the botanical extract is prepared from botanical leaves that are 2 years or younger.

14. The composition of claim 1, wherein the botanical extract is prepared from botanical leaves that are about 3-6 months old.

15. The composition of claim 1, wherein the botanical extract is prepared from yerba mate leaves.

16. A method of preparing a botanical extract, the method comprising:
combining botanical leaves with a first solvent to form a first mixture;
heating the first mixture;
filtering the first mixture to obtain a first liquid extract and a first residual material;
combining the first residual material with a second solvent to form a second mixture;
heating the second mixture;
filtering the second mixture to obtain a second liquid extract and a second residual material; and
combining and concentrating the first liquid extract and second liquid extract to obtain the botanical extract.

17. The method of claim 16, wherein the first solvent to botanical leaves has a ratio ranging from about 1:1 to about 20:1 in mL to grams.

18. The method of claim 16, wherein the botanical extract comprises saponins in an amount of at least 2.5%, caffeoylquinic acids in an amount of at least 20%, and caffeine in an amount of at least 4%.

19. A method of improving or treating a metabolic condition, comprising administering to a subject in need a composition comprising:
a botanical extract, comprising:
at least 2.5% saponins;
at least 20% polyphenols; and
at least 4% xanthines,
wherein the composition is formulated as a powder, tablet, capsule, or liquid.

20. The method of claim 19, wherein the composition is administered orally at least once daily.
